SEQUENCE LISTING (34 sequences)

SEQ ID NO: 1 - protein, 106 aa, Mus musculus
QIVLTQSPPIMSASLGEEITLTCSASSRVSYMLWYQQKSGTSPKLLIYSTSNLASGVPSRFSGSGSGTFYSLTISSVEAEDAADYYCHQWTSNPCTFGGGTKLEIK

SEQ ID NO: 2 - protein, 107 aa, Mus musculus
STVMTQTPKFLLVSAGDRVTITCKASQSVSNDVAWYQQKPGQSPKVLIYFASNRYTGVPDRFTGSGYGTDFTFTINTVQAEDLAVYFCQQDYSSPLTFGAGTKLELK

SEQ ID NO: 3 - protein, 106 aa, Mus musculus
QIVLSQSPAILSASPGEKVTMTCRASSSVSYMHWYQQRPGSSPKPWIYATSNLASGVPARFSGSGSGTSYYFTISRVEAEDAATYYCQQWNSNPPTHGGGTKLEIK

SEQ ID NO: 4 - protein, 106 aa, Mus musculus
QIVLSQSPAILSASPGEKVTMTCRASSSVSYMDWYQQKPGSSPKPWIYATSNLASGVPARFSGSGSGTSYSLTISRVEAEDTATYYCQQWTFNPPTFGSGTKLEIK

SEQ ID NO: 5 - protein, 106 aa, Mus musculus
QIVLSQSPAILSASPGEKVTMTCRASSSVSYMHWYQQKPGSSPKPWIYATSNLASGVPARFSGSGSGTSYSLTITRVEAEDAATYYCQQWTFNPPTFGAGTKLELK

SEQ ID NO: 6 - protein, 106 aa, Mus musculus
QIVLSQSPAILSSSPGEKVTMTCRASSSVSYMLWYQQKPGSSPEPWIYATSNLASGVPARFSGGGSGTSYSLTISRVEAEDAATYYCQQWTFNPPTFGGGTKLEIK

SEQ ID NO: 7 - protein, 107 aa, Mus musculus
DILLTQSPAILFVSPGERVSLSCRASQNIGTSIHWYQQRTNGSPRLLIKYASESFSGIPSRFSGSGSGTDFTLSINSVESEDIADYYCQQSNSWPFTFGSGTKLEIK

SEQ ID NO: 8 - protein, 108 aa, Mus musculus
EIILTQSPTTMAASPGEKITITCSASSSISSYYLRWYQQKPGFSPKVLIYRTSNLASGVPARFSGSGSGTSYSLTIGTMEAEDVATYYCQQGNTVPLTFGSGTKLEIK

SEQ ID NO: 9 - protein, 123 aa, Mus musculus
QVQLQQPGAELVKPGASVKMSCRASGYTFTNYNMHWIKQTPGQGLEWIGAIYPGSGDTSYNRKFKGKATLTADTSSSTAYMQFSSLTSADSAVYYCARFTYYYGGTYGAMDYWGQGTSVTVSL

SEQ ID NO: 10 - protein, 124 aa, Mus musculus
QIQLVQSGPELKKPGETVKISCKASGYTFTNFGVNWVKQAPGKGLKWMGWINTYTGEPSYADDFKGRFAFSLEASANTAYLQINNLKNDDMSTYFCTRRTNYYGTSYYYAMDYWGQGTSVTVSS

SEQ ID NO: 11 - protein, 119 aa, Mus musculus
QVQLQQPGAELVKPGASVKMSCKASGYTFTSYNIHWVKQTPGQGLEWIGAIYPGNGDTSYNQKFKGKATLTSDKSSSTAYMQLSSLTSEDSAVYYCARMSTMITGFDYWGQGTTLTVSS

SEQ ID NO: 12 - protein, 122 aa, Mus musculus
QVQLQQPGAELVKPGASVKMSCKASGFTFTSYNLHWVKQTPGQGLEWIGAIYPGSGDTSYNQQFKGKATLTADKSSNTAYMQLNSLTSEDSAVYCCARSAMISTGNWYFDYWGQGTTLTVSS

SEQ ID NO: 13 - protein, 121 aa, Mus musculus
QVQLQQPGAELVKPGASVKMSCKASGFTFTSYNMHWVKQTPGQGLEWIGGIYPGNGDTSYNQKFKGKATLTADKSSSTAYMQLSSLTSEDSAVYYCARFYYYGSMGAMDYWGQGTSVTVSS

SEQ ID NO: 14 - protein, 122 aa, Mus musculus
QVQLQQPGAELVKPGASVKMSCKASGYTFTTYNLHWVKQTPGQGLEWIGGIYPGNGDTSYNQKFKVKATLTADKSSNTAYMQLSSLTSEDSAVYYCARWIYYGNYEGTLDYWGQGTSVTVSS

SEQ ID NO: 15 - protein, 116 aa, Mus musculus
QVQLQQSGAELAKPGASVKMSCKASGYTFTSYWMHWVKQRPGQGLEWIGYITPSTGYTDYNKKFKDKATLTADRSSSTAYMHLSSLTSEDSAVYYCARSGPYFDVWGAGTTVTVSS

SEQ ID NO: 16 - protein, 119 aa, Mus musculus
QVQLQQPGAELVKPGASVKVSCKASGFTFTSYNLHWVKQTPGQGLVWIGAIYPGNGDTSYNQKFRGKATLTADISSSTAYMQLSSLTSEDSAVYYCARYYYGYDAMDYWGQGTSVTVSS

SEQ ID NO: 17 - protein, 107 aa, Artificial
  L chain V region sequence of humanized antibody abb 1791
STVMTQSPDSLAVSLGERATINCKSSQSVSNDVAWYQQKPGQSPKVLIYFASNRYSGVPDRFSGSGYGTDFTLTISSLQAEDVAVYFCQQDYSSPLTFGAGTKLEIK

SEQ ID NO: 18 - protein, 107 aa, Artificial
  L chain V region sequence of humanized antibody fra 1791
STVMTQSPSFLSASVGDRVTITCKASQSVSNDVAWYQQKPGQSPKVLIYFASNRYTGVPDRFSGSGYGTDFTLTISSLQAEDVAVYFCQQDYSSPLTFGAGTKLEIK

SEQ ID NO: 19 - protein, 107 aa, Artificial
  L chain V region sequence of humanized antibody sdr 1791
STVMTQSPDSLAVSLGERATINCKSSQSNSNDVAWYQQKPGQSPKVLIYFASNRYSGVPDRFSGSGYGTDFTLTISSLQAEDVAVYFCQQDYSSPLTFGAGTKLEIK

SEQ ID NO: 20 - protein, 107 aa, Artificial
  L chain V region sequence of humanized antibody Ven 1791
STVMTQSPDSLAVSLGERVTINCKASQSVSNDVAWYQQKPGQSPKVLIYFASNRYTGVPDRFSGSGYGTDFTFTISSVQAEDVAVYFCQQDYSSPLTFGAGTKLELK

SEQ ID NO: 21 - protein, 124 aa, Artificial
  H chain V region sequence of humanized antibody abb 1791
QIQLVQSGSELKKPGASVKVSCKASGYTFTNFGVNWVRQAPGKGLEWMGWINTYTGEPSYAQGFTGRFVFSLDASVSTAYLQISSLKAEDTATYFCTRRTNYYGTSYYYAMDYWGQGTTVTVSS

SEQ ID NO: 22 - protein, 124 aa, Artificial
  H chain V region sequence of humanized antibody fra 1791
QIQLVQSGSELKKPGASVKVSCKASGYTFTNFGVNWVKQAPGKGLKWMGWINTYTGEPSYADDFKGRFAFSLDASASTAYLQISSLKAEDMATYFCTRRTNYYGTSYYYAMDYWGQGTTVTVSS

SEQ ID NO: 23 - protein, 124 aa, Artificial
  H chain V region sequence of humanized antibody sdr 1791
QIQLVQSGSELKKPGASVKVSCKASGYTFTNFGVNWVRQAPGKGLKWMGWINTYTGEPSYAQGFTGRFAFSLDASVSTAYLQISSLKAEDTATYFCTRRTNYYGTSYYYAMDYWGQGTTVTVSS

SEQ ID NO: 24 - protein, 124 aa, Artificial
  H chain V region sequence of humanized antibody Ven 1791
QIQLVQSGPELKKPGASVKISCKASGYTFTNFGVNWVKQAPGKGLKWMGWINTYTGEPSYADDFKGRFAFSLDASVSTAYLQISSLKAEDTSTYFCTRRTNYYGTSYYYAMDYWGQGTTVTVSS

SEQ ID NO: 25 - DNA, 35 bp, Artificial sequence
  primer
aatgcggccgccaccatgacaacacccagaaattc

SEQ ID NO: 26 - DNA, 29 bp, Artificial sequence
  primer
gctctagattaaggagagctgtcattttc

SEQ ID NO: 27 - protein, 107 aa, Artificial
  L chain V region sequence of humanized antibody Ven 1782
DILLTQSPATLSLSPGERATLSCRASQNIGTSIHWYQQKPGQSPRLLIKYASESFSGIPSRFSGSGSGTDFTLTISSLEPEDFADYYCQQSNSWPFTFGSGTKLEIK

SEQ ID NO: 28 - protein, 107 aa, Artificial
  L chain V region sequence of humanized antibody abb 1782
DIVLTQSPATLSLSPGERATLSCRASQNIGTSIHWYQQKPGQSPRLLIKYASESFTGIPSRFSGSGSGTDFTLTISSLEPEDFADYYCQQSNSWPFTFGSGTKLEIK

SEQ ID NO: 29 - protein, 107 aa, Artificial
  L chain V region sequence of humanized antibody sdr 1782
DILLTQSPATLSLSPGERATLSCRASQNVGTSLHWYQQKPGQSPRLLIKYASERFTGIPSRFSGSGSGTDFTLTISSLEPEDFADYYCQQSNSWPFTFGSGTKLEIK

SEQ ID NO: 30 - protein, 107 aa, Artificial
  L chain V region sequence of humanized antibody fra 1782
DILLTQSPATLSVSPGERATLSCRASQNIGTSIHWYQQRPGQSPRLLIKYASESFSGIPSRFSGSGSGTDFTLTINSLQPEDIADYYCQQSNSWPFTFGSGTKLEIK

SEQ ID NO: 31 - protein, 116 aa, Artificial
  H chain V region sequence of humanized antibody Ven 1782
QVQLVQSGAELKKPGASVKVSCKASGYTFTSYWMHWVKQAPGQGLEWIGYITPSTGYTDYNKKFKDKATLTADRSSSTAYMELSSLRSEDTAVYYCARSGPYFDVWGAGTTVTVSS

SEQ ID NO: 32 - protein, 116 aa, Artificial
  H chain V region sequence of humanized antibody abb 1782
QVQLVQSGAEVKKPGASVKVSCKASGYTFTSYWMHWVRQAPGQGLEWMGYITPSTGYTDYNQKFQGRVTLTADRSSSTAYMELSSLRSEDTAVYYCARSGPYFDVWGAGTTVTVSS

SEQ ID NO: 33 - protein, 116 aa, Artificial
  H chain V region sequence of humanized antibody sdr 1782
QVQLVQSGAEVKKPGASVKVSCKASGYTFTSYWMHWVKQAPGQGLEWIGYITPSTGYTDYNQKFQGKATLTADRSSSTAYMELSSLRSEDTAVYYCARSGPYFDVWGAGTTVTVSS

SEQ ID NO: 34 - protein, 116 aa, Artificial
  H chain V region sequence of humanized antibody fra 1782
QVQLVQSGAEVKKPGASVKVSCKASGYTFTSYWMHWVKQAPGQGLEWIGYITPSTGYTDYNKKFKDKATLTADRSSSTAYMELSSLRSEDTAVYYCARSGPYFDVWGAGTTVTVSS

(19) United States
(12) Patent Application Publication
(10) Pub. No.: US 2011/0263825 A1
(43) Pub. Date: Oct. 27, 2011

(54) ANTI-CD20 MONOCLONAL ANTIBODIES

(75) Inventors: Susumu Uchiyama, Osaka (JP); Kiichi Fukui, Osaka (JP)

(73) Assignee: OSAKA UNIVERSITY, Suita-shi, Osaka (JP)

(21) Appl. No.: 12/674,783

(22) PCT Filed: Sep. 6, 2007
(86) PCT No.: PCT/JP2007/067444

§ 371 Date: Feb. 23, 2010

(51) Int. Cl. C07K 16/28; C12P 21/08

(52) U.S. Cl. 530/387.3; 435/70.21

(57) ABSTRACT

It is intended to provide a monoclonal antibody having a growth inhibitory activity against a cell having a human CD20 antigen which is produced by using, as immunogens, a human B cell line expressing the human CD20 antigen and a cell line originating in a non-human animal, which is different from an animal to be immunized and has been transformed with human CD20 DNA, and a monoclonal antibody obtained by chimerization or humanization of the above-described monoclonal antibody. These monoclonal antibodies show biological activities suitable for using as drugs.

### TECHNICAL FIELD

The present invention relates to an anti-CD20 monoclonal antibody.

### BACKGROUND ART

CD20 is a protein not containing sugar chains, which is expressed on the cellular surface of human B-lymphocytes. CD20 is expressed on many malignant tumor B-cells, in addition to normal B-cells in the peripheral blood, spleen, tonsils, and bone marrow. Epitopes to which monoclonal antibodies directed to CD20 bind are extremely highly varied and a wide variety of biological responses have been reported. Furthermore, there have been many reports of monoclonal antibodies recognizing CD20. Among them, rituximab is a chimerized mouse/human monoclonal antibody (C2B8), which is derived from a mouse antibody 2B8 obtained by immunization of an SB cell strain, a type of human B-cell (see Patent Documents 1 and 2). Rituximab has been actually used under the name Rituxan® as a therapeutic agent for the treatment of low malignant non-Hodgkin's lymphoma (NHL). Since then, it has been further reported that Rituxan is effective against many immune diseases related to B-cells, for example, malignant tumors, such as chronic lymphocytic leukemia (CCL), autoimmune diseases involving pathogenic autoantibodies, such as autoimmune hemolyticanemia and idiopathic thrombocytopenic purpura (ITP), and inflammatory diseases, such as rheumatoid arthritis (RA) and multiple sclerosis (see Non-Patent Documents 1 to 4).

It has been reported that rituximab binds to human complements, resulting in lysis of B-cells of the lymphatic linenage by complement-dependent cytotoxicity (CDC) (see Non-Patent Document 5), and that rituximab displays activity in assays for antibody-dependent cell-mediated cytotoxicity (ADCC) and growth inhibiting activity and apoptosis induction in tritiated thymidine incorporation assays (see Non-Patent Document 6).

Chimera molecules with molecules from different animal species are antigenic and therefore are generally not desirable as therapeutic agents, while it has been believed that anti-CD20 antibodies, including rituximab, are not antigenic since they target and eliminate all types of B-cells, including normal cells. However, it has been reported that there are cases where rituximab induces neutralizing antibodies, albeit at several percent, during therapy, and the possibility of inducing neutralizing antibodies may further increase depending on its dosage amount and duration. In addition, there is an increased focus on problems associated with antigenicity, in the course of extending the target to be treated from B-cell lymphomas to RA, IT, and MS. For these reasons, there has been a recent need for human antibodies or humanized antibodies containing sequences more similar to human sequences.

Chimerized antibodies entail the problem that they have relatively short half-lives in blood. The β half-life (β ½) of chimerized mouse/human antibodies, including rituximab, is no more than 3 to 4 days. The efficacy in clinical trials of rituximab against low malignant NHL has been reported to be a little less than 50% (see Non-Patent Document 7). Furthermore, there is a problem that amounts of rituximab to be administered in NHL therapy are increased, since the dissociation constant (Kd value) of rituximab for the CD20 antigen is 5.2 nM and the binding affinity is not very high (see Non-Patent Document 8).
[Patent Document 1]: International Publication No. WO 94/11026 pamphlet
[Patent Document 2]: U.S. Pat. No. 5,736,137 specification
[Non-Patent Document 1]: Coiffier B et al., Blood 1998; 92:1297-32
[Non-Patent Document 2]: Edward J C at al., Rheumatology (Oxford) 2001; 40:205-11
[Non-Patent Document 3]: Zaja F at al., Heamatologica 2002; 87:189-95
[Non-Patent Document 4]: Perrotta S at al., Br J Haematol 2002; 116:465-7
[Non-Patent Document 5]: Reff et al., Blood 1994; 83: 435-445
[Non-Patent Document 6]: Maloney at al., Blood 1996; 88: 637a
[Non-Patent Document 7]: IDEC Pharmaceuticals Corporation News Release, Dec. 8, 1998
[Non-Patent Document 8]: Mitchell E R at al., Blood 1994; 82:435-445

### DISCLOSURE OF THE INVENTION

### Problems to be Solved by the Invention

In view of the problems discussed above, the present invention has the principal object of providing an anti-CD20 monoclonal antibody displaying biological activities more suitable as pharmaceutical use.

### Means to Solve the Problems

In order to attain the above object, the present inventors have performed diligent research to prepare a monoclonal antibody displaying high binding affinity to human CD20 molecule in its natural state, thereby obtaining an anti-CD20 monoclonal antibody having excellent characteristics. In consequence, the inventors have found that high-affinity monoclonal antibodies displaying excellent biological activities can be obtained by using, as immunogens, SB or Raji cells, which are of a B-cell strain thought to contain a high density of CD20 antigen, combined with non-human animal cells modified using genetic recombination to express a large amount of CD20 on the cellular membrane, thereby having arrived at the present invention.

In other words, the present invention provides the following:

(1) a humanized anti-CD20 monoclonal antibody, comprising a combination of the L-chain set forth in any one of SEQ ID Nos: 27 to 30 and the H-chain set forth in any one of SEQ ID Nos: 31 to 34;
(2) the humanized anti-CD20 monoclonal antibody according to the above-described (1), comprising a combination of the L-chain set forth in SEQ ID No: 30 and the H-chain set forth in SEQ ID No: 34;
(3) the humanized anti-CD20 monoclonal antibody according to the above-described (1), comprising a combination of the L-chain set forth in SEQ ID No: 30 and the H-chain set forth in SEQ ID No: 31;
(4) the humanized anti-CD20 monoclonal antibody according to the above-described (1), comprising a combination of the L-chain set forth in SEQ ID No: 29 and the H-chain set forth in SEQ ID No: 33;
(5) the humanized anti-CD20 monoclonal antibody according to the above-described (1), which is produced by a cell selected from the group consisting of cells which have been deposited under the accession numbers FERM ABP-10907, FERM ABP-10906, and FERM ABP-10905, with the International Patent Organism Depositary of the National Institute of Advanced Industrial Science and Technology;
(6) a method for producing a humanized anti-CD20 monoclonal antibody, which comprises culturing a cell selected from the group consisting of cells which have been deposited under the accession numbers FERM ABP-10907, FERM ABP-10906, and FERN ABP-10905, with the International Patent Organism Depositary of the National Institute of Advanced industrial Science and Technology;
(7) a therapeutic agent for the treatment of B-cell mediated diseases, comprising as an active ingredient the humanized anti-CD20 monoclonal antibody according to any one of the above-described (1) to (6).

### Effects of the Invention

The present invention provides a monoclonal antibody, in particular, humanized monoclonal antibody, and a method for producing the same, which displays a high binding affinity to an extracellular epitope of a CD20 antigen and possess biological activities, such as inhibitory activity of cell growth, and which is suitable as pharmaceutical use. The present invention also provides a therapeutic agent for the treatment of diseases involving B-cells, comprising such a monoclonal antibody.

### BRIEF DESCRIPTION OF THE DRAWINGS

FIG. 1 is a restriction map showing the structure of pNOW-Ab, a vector for expressing a recombinant antibody.

FIG. 2 is a restriction map showing the structure of pNOW, a vector for expressing a protein.

FIG. 3a is a graph showing the results of an apoptosis test.

FIG. 3b is a graph showing the results of an apoptosis test.

FIG. 3c is a graph showing the results of an apoptosis test.

FIG. 3d is a graph showing the results of an apoptosis test.

FIG. 4a is a graph showing the relationship between antibody concentration and ADCC (using RAJI cells).

FIG. 4b is a graph showing h relationship between antibody concentration and ADCC (using WIL2 cells).

FIG. 4c is a graph showing the relationship between antibody concentration and ADCC (using SU-DHL4 cells).

FIG. 4d is a graph showing the relationship between antibody concentration and ADCC (using RC-K8 cells).

FIG. 5a is a graph showing the relationship between E:T ratio and ADCC (using RAJI cells).

FIG. 5b is a graph showing the relationship between E:T ratio and ADCC (using WIL2 cells).

FIG. 5c is a graph showing the relationship between E:T ratio and ADCC (using SU-DHL4 cells).

FIG. 5d is a graph showing the relationship between E:T ratio and ADCC (using RC-K8 cells).

FIG. 6a is a graph showing the results of a CDC test (using RAJI cells).

FIG. 6b is a graph showing the results of a CDC test (using WIL2 cells).

FIG. 6c is a graph showing the results of a CDC test (using SU-DHL4 cells).

FIG. 6d is a graph showing the results of a CDC test (using RC-K8 cells).

FIG. 7a is a graph showing the results of apoptosis experiments using a mouse antibody. The numbers on the right side in the figure represent the concentration of the antibody (μg/ml), and + and − represent the presence and the absence of crosslinking with a goat antibody, respectively (these representations apply in FIG. 7a to FIG. 7d). The cell used is RAJI cell.

FIG. 7b is a graph showing the results of apoptosis experiments using a mouse antibody. The cell used is WIL2 cell.

FIG. 7c is a graph showing the results of apoptosis experiments using a mouse antibody. The cell used is SU-DHL4 cell.

FIG. 7d is a graph showing the results of apoptosis experiments using a mouse antibody. The cell used is RC-K8 cell.

FIG. 8a is a graph showing the results of apoptosis experiments using a humanized antibody. The numbers on the right side in the figure represent the concentration of the antibody (μg/ml), and + and − represent the presence and the absence of crosslinking with a goat antibody, respectively (these representations apply in FIGS. 8a to 8d). The cell used is RAJI cell.

FIG. 8b is a graph showing the results of apoptosis experiments using a humanized antibody. The cell used is WIL2 cell.

FIG. 8c is a graph showing the results of apoptosis experiments using a humanized antibody. The cell used is SU-DHL4 cell.

FIG. 8d is a graph showing the results of apoptosis experiments using a humanized antibody. The cell used is RC-K8 cell.

FIG. 9a is a graph showing the ratio of early apoptosis using a humanized antibody. The value in the case of adding no antibody is set to 1. The numbers on the right side in the figure represent the concentration of the antibody (μg/ml), + and − represent the presence and the absence of crosslinking with a goat antibody, respectively (these representations apply in FIGS. 9a to 9d). The cell used is RAJI cell.

FIG. 9b is a graph showing the ratio of early apoptosis using a humanized antibody. The cell used is WIL2 cell.

FIG. 9c is a graph showing the ratio of early apoptosis using a humanized antibody. The cell used is SU-DHL4 cell.

FIG. 9d is a graph showing the ratio of early apoptosis using a humanized antibody. The cell used is RC-K8 cell.

FIG. 10a is a graph showing CDC activities of humanized antibodies and chimerized antibodies. The cell used is Raji cell.

FIG. 10b is a graph showing CDC activities of humanized antibodies and chimerized antibodies. The cell used is SU-DHL4 cell.

FIG. 10c is a graph showing CDC activities of humanized antibodies and chimerized antibodies. The cell used is WiL2 cell.

FIG. 10d is a graph showing CDC activities of humanized antibodies and chimerized antibodies. The cell used is RC-K8 cell.

FIG. 11a is a graph showing the relationship between humanized antibody concentration and ADCC. The cell used is RAJI cell.

FIG. 11b is a graph showing the relationship between humanized antibody concentration and ADCC. The cell used is SU-DHL4 cell.

FIG. 11c is a graph showing the relationship between humanized antibody concentration and ADCC. The cell used is WiL2 cell.

FIG. 11d is a graph showing the relationship between humanized antibody concentration and ADCC. The cell used is RC-K8 cell.

FIG. 12a is a graph showing the results of apoptosis experiments using a humanized antibody. (XL) represents the presence of crosslinking with a secondary antibody (goat anti-human antibody). The cell used is RC-K8 cell.

FIG. 12b is a graph showing the results of apoptosis experiments using a humanized antibody. (XL) represents the presence of crosslinking with a secondary antibody (goat anti-human antibody). The cell used is SU-DHL4 cell.

FIG. 12c is a graph showing the results of apoptosis experiments using a humanized antibody. (XL) represents the presence of crosslinking with a secondary antibody (goat anti-human antibody). The cell used is RAJI cell.

FIG. 12d is a graph showing the results of apoptosis experiments using a humanized antibody. (XL) represents the presence of crosslinking with a secondary antibody (goat anti-human antibody). The cell used is WIL2NS cell.

FIG. 13a is a graph showing the results of apoptosis experiments using a humanized antibody. (XL) represents the presence of crosslinking with a secondary antibody (goat anti-human antibody). The cell used is RC-K8 cell. The activity of inducing apoptosis with no added antibody is set to 1.

FIG. 13b is a graph showing the results of apoptosis experiments using a humanized antibody. (XL) represents the presence of crosslinking with a secondary antibody (goat anti-human antibody). The cell used is SU-DHL4 cell. The activity of inducing apoptosis with no added antibody is set to 1.

FIG. 13c is a graph showing the results of apoptosis experiments using a humanized antibody. (XL) represents the presence of crosslinking with a secondary antibody (goat anti-human antibody). The cell used is RAJI cell. The activity of inducing apoptosis with no added antibody is set to 1.

FIG. 13d is a graph showing the results of apoptosis experiments using a humanized antibody. (XL) represents the presence of cross linking with a secondary antibody (goat anti-human antibody). The cell used is WIL2NS cell. The activity of inducing apoptosis with no added antibody is set to 1.

FIG. 14a is a graph showing CDC activities of humanized antibodies. The cell used is RC-K8 cell.

FIG. 14b is a graph showing CDC activities of humanized antibodies. The cell used is SU-DHL4 cell.

FIG. 15a is a graph showing ADCC activities of humanized antibodies. The cell used is RC-K8 cell.

FIG. 15b is a graph showing ADCC activities of humanized antibodies. The cell used is SU-DHL4 cell.

### DESCRIPTION OF ABBREVIATIONS

Pcmv: Cytomegalovirus promoter
PAbgh: Poly A addition signal of bovine growth hormone gene
Psvd: Enhancer-deleted simian virus 40 promoter
DHFR: cDNA for mouse dihydrofolate reductase
PAsv: Poly A addition signal from simian virus 40
PBR322ori: E. coli origin of replication
Amp^{r}: E. coli selective marker (ampicillin resistance)
Neo^{r}: Mammalian cell selective marker (G418 resistance)
INrbg: Rabbit β-globin intron
SP1: Antibody light-chain signal peptide
VL: cDNA for a light-chain variable region of an antibody
Cκ: cDNA for a κ light-chain constant region of an antibody
SPh: Antibody light-chain signal peptide
Vh: cDNA for a light-chain variable region of an antibody
Cγ1: cDNA for a γ1 heavy-chain constant region of an antibody

### BEST MODE FOR CARRYING OUT THE INVENTION

In this specification, an “antibody” includes not only a whole antibody, but also fragments thereof displaying binding affinities to its antigen which are comparable to that of the whole antibody, such as fragments containing the variable region of the original whole antibody (for example, Fab, F(ab′)₂, and the like).

Monoclonal antibodies according to the present invention are monoclonal antibodies which display high affinities to human CD20 antigen and have excellent biological activities, and include mouse-derived monoclonal antibodies, and chimerized and humanized variants thereof.

According to a preferred, first embodiment of the present invention, there is provided a monoclonal antibody having a growth inhibiting activity against cells expressing human CD20 antigen and a high affinity to human CD20 antigen, with a dissociation constant (Kd value) for Raji cells (floating cells) of not more than one half of the Kd value of the mouse antibody 2B8, from which Rituximab is derived, preferably with a Kd value between 1.7 and 3.39 nM.

There is no particular limitation on methods for measuring the dissociation constant (Kd value), as long as these methods allow the Kd value to be measured with respect to floating cells. In this specification, however, the dissociation constant is taken to be one obtained by the method described hereinafter in the Examples.

It is preferable that growth inhibiting activity of the antibody of the present invention against cells expressing human CD20 antigen is greater than that of 2B8. The growth inhibiting activity preferably is a growth inhibiting activity with respect to in vitro cultures of cells expressing human CD20 antigen, in the absence of peripheral blood monocytes, more preferably, a growth inhibiting activity due to apoptosis induction.

Measurements of the growth inhibiting activity as described above can be made, for example, using the method described in Miyamoto T, Min W, Lillehoj H S. Avian Dis., 2002 January-March; 46(1):10-6.

Specific examples of monoclonal antibodies according to the first embodiment of the present invention include a mouse-derived monoclonal antibody wherein the amino acid sequence of the L-chain variable region and the amino acid sequence of the H-chain variable region are set forth in SEQ ID Nos: 1 and 9, SEQ ID Nos: 2 and 10, or SEQ ID Nos: 3 and 11, respectively, and chimerized or humanized variants thereof.

Chimerization can be carried out, for example, by fusing an amino acid sequence from the variable region of a mouse-derived monoclonal antibody with an amino acid sequence from the constant region of human immunoglobulin, according to known methods as described in Ishida T, Imai K, Nippon Rinsho, Vol. 60, No. 3, 2002-3:439-444.

Humanization can be carried out, for example, by using the CDR amino acid sequence of the variable region of a mouse-derived monoclonal antibody and the amino acid sequence of human immunoglobulin, according to known methods as described in Ishida T, Imai K, Nippon Rinsho, Vol. 60, No. 3, 2002-3:439-444, and also in Eduardo A. Padlan, Molecular Immunology, Vol. 28-4/5, pp. 489-498, 1991; Eduardo A. Padlan et. al., The EASES Journal, vol. 9, pp. 133-139; and Tai to Wu, Elvin A. Kabat, Molecular Immunology, Vol. 29-9, pp. 1141-1146, 1992.

When chimerization or humanization is carried out, one may combine, in any combination, an amino acid sequence from the L-chain variable region and an amino acid sequence from the H-chain variable region of a plurality of mouse monoclonal antibodies. Examples include, for example, chimerized anti-CD20 monoclonal antibodies combining the L-chain which has a chimerized amino acid sequence from the amino acid sequence of the variable region of a mouse-derived monoclonal antibody set forth in any one of SEQ ID Nos: 1 to 3 and the H-chain which has a chimerized amino acid sequence from the amino acid sequence of the variable region of a mouse-derived monoclonal antibody set forth in SEQ ID Nos: 9 to 11; and humanized anti-CD20 monoclonal antibodies combining the L-chain which has a humanized amino acid sequence of the CDR amino acid sequence of the variable region of a mouse-derived monoclonal antibody set forth in any one of SEQ ID Nos: 1 to 3 and the H-chain which has a humanized amino acid sequence of the CDR the amino acid sequence of the variable region of a mouse-derived monoclonal antibody set forth in any one of SEQ ID Nos: 9 to 11.

An antibody according to a preferred, second embodiment of the present invention is a mouse-derived chimerized or humanized monoclonal antibody which has a dissociation constant (Kd value) for Raji cells, which are floating cells, of not more than one eighth of the Kd value of 2B8.

It is well known that when antibodies having high affinity for human CD20 antigen and containing, in particular, human IgG1 or IgG3 sequence, or an human Fc sequence modified therefrom (including chimerized or humanized versions of mouse-derived monoclonal antibodies) bind to CD20 on the cell surface, they induce activation of effector cells via FcγRIII (CD16) on NM cells, resulting in antibody-dependent cell-mediated cytotoxicity (ADCC). It is well known that when antibodies having high affinity for humanized CD20 antigen and containing, in particular, human IgG or IgM sequence, or a human Fc sequence modified form (including chimerized or humanized versions of mouse-derived monoclonal antibodies) bind to CD20 on the cell surface, they induce activation of complements, resulting in complement-dependent cytotoxicity (CDC).

Therefore, antibodies according to the second embodiment of the present invention can be expected to have a low or undetected growth-inhibiting activity and to display ADCC or CDC.

Specific examples of antibodies according to the second embodiment of the present invention include antibodies wherein the amino acid sequence of the L-chain variable region and the amino acid sequence of the H-chain variable region are set forth in SEQ ID Nos: 4 and 12, SEQ ID Nos: 5 and 13, SEQ ID Nos: 6 and 14, SEQ ID Nos: 7 and 15, or SEQ ID Nos: 8 and 16, respectively.

Chimerization or humanization can be carried out by similar methods as in the antibodies according to the first embodiment. One may combine, in may combination, an amino acid sequence from the L-chain variable region and an amino acid sequence from the H-chain variable region of a plurality of mouse monoclonal antibodies. Examples include, for example, chimerized anti-CD20 monoclonal antibodies combining the L-chain which has a chimerized amino acid sequence from the amino acid sequence of the variable region of a mouse-derived monoclonal antibody set forth in any one of SEQ ID Nos: 4 to 8 and the H-chain which has a chimerized amino acid sequence from the amino acid sequence of the variable region of a mouse-derived monoclonal antibody set forth in SEQ ID Nos: 12 to 16; and humanized anti-CD20 monoclonal antibodies combining the L-chain which has a humanized amino acid sequence of the CDR amino acid sequence of the variable region of a mouse-derived monoclonal antibody set forth in any one of SEQ ID Nos: 4 to 8 and the H-chain which has a humanized amino acid sequence of the CDR amino acid sequence of the variable region of a mouse-derived monoclonal antibody set forth in any one of SEQ ID Nos: 12 to 16.

An antibody according to a preferred, third embodiment of the present invention belongs to a group of humanized monoclonal antibodies which are not limited by a specific dissociation constant (Kd value) for 2B8, including humanized monoclonal antibodies which are effective against cells on which rituximab has no effect.

Examples of such antibodies include humanized anti-CD20 monoclonal antibodies combining the L-chain set forth in SEQ ID No: 18 and the H-chain set forth in SEQ ID No: 24, the L-chain set forth in SEQ ID No: 18 and the H-chain set forth in SEQ ID No: 22, the L-chain set forth in SEQ ID No: 19 and the H-chain set forth in SEQ ID No: 22, and the L-chain set forth in SEQ ID No: 19 and the H-chain set forth in SEQ ID No: 23.

Mouse-derived monoclonal antibodies according to the present invention or mouse-derived monoclonal antibodies which can be used in the preparation of antibodies according to the present invention can be prepared, for example, by selecting a clone producing a monoclonal antibody having desired properties from hybridoma clones obtained by screening with the methods described below.

As sensitizing antigen (immunogen) are used, for example, SB or Raji cells which are CD20 expressing cells and CHO cells which have been transformed by recombinant techniques, for example, using a commercially available DNA for CD20 (or fragments thereof having the same effect) (CHO/CD20), so as to express CD20 on the cell surface. The initial immunization, booster immunization(s), and the final immunization are administered such that the initial immunization and the booster immunization(s) are either carried out at least once using a cell strain which presents the sensitizing antigen and is derived from an animal belonging to a different order from the subject animal for immunization or carried out at least once using a cell strain which presents the sensitizing antigen on the surface of the cell membrane by genetic recombination and is derived from an animal belonging to the same order as the subject animal for immunization, and the final immunization is carried out using the other cell strain.

Other conditions may be similar to conditions used in methods for generating hybridomas producing usual monoclonal antibodies. Hybridomas producing monoclonal antibodies are generated by (1) immunization of animals to be immunized (2) preparation of lymphocytes from the immunized animals, (3) preparation of parent cells, (4) cell fusion of the lymphocytes and the parent cells, and (5) screening and cloning, according to known methods (see, for example, Monokuronaru kotai, Seikagaku Jikkenho [Monoclonal Antibody, Biochemical Experiment Methods], Ailsa M. Campbell (ed.), Toshiaki OSAWA (trans.), Tokyo Kagaku Dojin (1989)).

Methods for preparing monoclonal antibodies using cloned hybridomas can be similar to conventional methods for preparing monoclonal antibodies, except for employing hybridomas prepared using the method of hybridoma production according to the present invention. Large-scale production can be effected, for example, using methods by which the antibody is produced by cell culturing or as mouse ascites. Production of chimerized or humanized antibodies can be performed by preparing a gene coding for the chimerized or humanized antibody, inserting the gene into an expression vector, and expressing the expression vector in a suitable cell.

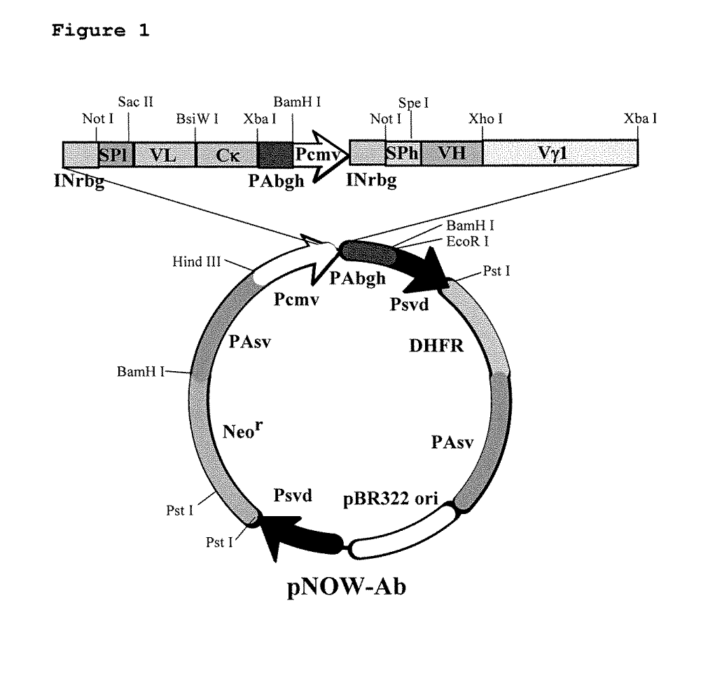

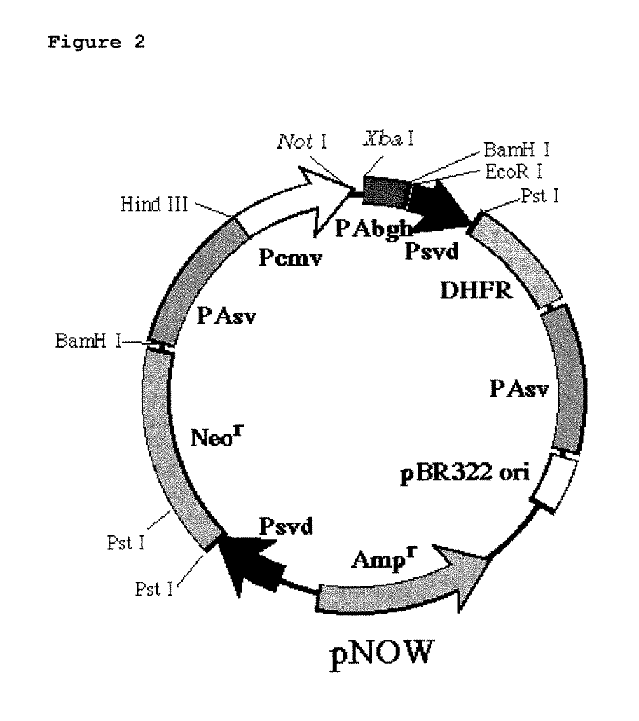

For example, genes for the L-chain variable region and the H-chain variable region can be chimerized using genes for the L-chain constant region and the H-chain (K) constant region of human immunoglobulin and inserted into a high expression vector for CHO cell. Although commercially available vector systems for the production of recombinant antibodies may be employed, it is possible to use vectors constructed in pNOW-ab, a dimer high-expression vector containing multicloning sites (MCSs) for both the L-chain and the H-chain, which is based on pNOW, a high expression vector for mammalian cells (Japanese Patent No. 3,582,965). Restriction maps showing the structure of these vectors are shown in FIG. 1 and FIG. 2. CHO cells are transfected with an expression vector containing the chimerized antibody gene, and then highly-productive clones are selected. Antibodies are produced from these clones using usual methods.

Antibodies according to the first embodiment of the present invention display relative high binding affinities, as compared to rituximab, and high growth-inhibiting activities, preferably, due to apoptosis induction, and thus chimerized or humanized antibodies of these antibodies can be used as an active ingredient of therapeutic agents for malignant tumors of B-cells and diseases involving B-cells. Furthermore, antibodies according to the second and third embodiments of the present invention are thought to display complement-dependent cytotoxicity (CDC) or antibody-dependent cell-mediated cytotoxicity (ADCC) against cells expressing human CD20 antigen, and thus can be used as an active ingredient of therapeutic agents for malignant tumors of B-cells and immune diseases involving B-cells. Therefore, the present invention also provides therapeutic agents for diseases involving B-cells which have as an active ingredient the chimerized or humanized antibodies of the invention.

In addition, among the antibodies according to the present invention are antibodies that do not need an antibody requiring crosslinking with a secondary antibody in the induction of apoptosis, and these antibodies are suitable for pharmaceutical use, since antibodies capable of inducing apoptosis by themselves do not require any secondary antibody.

Furthermore, in the present invention, two or more types of antibodies according to the present invention can be combined.

Diseases involving B-cells include, but are not limited to, for example, non-Hodgkin's lymphoma, chronic lymphocytic leukemia, acute lymphocytic leukemia, rheumatoid arthritis, autoimmune hemolyticanemia, idiopathic thrombocytopenic purpura, systemic lupus erythematosus, anti-phospholipid antibody syndrome, Sjogren syndrome, Crohn's disease, scleroderma, multiple sclerosis, and others.

The therapeutic agents can be produced by techniques known for pharmaceutical preparations. There is not particular limitation on other formulating ingredients. Dosages and the like can be determined by reference to known Rituxan.

In further embodiments, the present invention provides a humanized anti-CD20 monoclonal antibody combining the L-chain set forth in any one of SEQ ID Nos: 27 to 30 and the H-chain set forth in any one of SEQ ID Nos: 31 to 34 (referred to herein as antibody 1782, antibody of the series 1782, or the like). Of these antibodies, exemplified as preferable antibodies are humanized anti-CD20 monoclonal antibodies in which the L-chain set forth in SEQ ID No: 30 and the H-chain set forth in SEQ ID No: 34 are combined (clone ff); the L-chain set forth in SEQ ID No: 30 and the H-chain set forth in SEQ ID No: 31 are combined (clone fv); and the L-chain set forth in SEQ ID No: 29 and the H-chain set forth in SEQ ID No: 33 are combined (clone ss). These humanized antibodies, which are also derived from mouse, can be humanized as described above.

The series 1782 of humanized antibodies has weak activity for inducing apoptosis by themselves, but display, under crosslinking conditions, apoptosis inducing activity equal to or higher than that of Rituxan (c2B8). Humanized antibody 1782 displays CDC activity not only against RAJI, WIL2NS, and SU-DHL4 cells, but also against RC-K8 cells which are of a Rituxan-resistant strain. In particular, as detailed in the Examples, clones ff and fv of the series 1782 have high CDC activities and display significantly greater CDC activities against SU-DHL4 cells than Rituxan. Putting together the results of Kd values and CDC activities, the clone ff of the series 1782 is preferable in that it has the highest affinity, a relatively high CDC activity, and a CDC activity also against the strain RC-K8, a Rituxan-resistant strain. The clone fv of the series 1782 is also preferable in that it has the highest CDC activity and in addition, is effective also on the strain RC-K8, a Rituxan-resistant strain. In particular, since the clone ff of the series 1782 has a very high affinity, it is possible to RI label it to employ it for missile therapy of B-cell involving diseases. Furthermore, the clone fv of the series 1782 is preferable in that it has a high CDC activity against SU-DHL4 cells, and the clone ss is preferable in that it has a high ADCC activity against RC-K8 cells.

The series 1782 of humanized anti-CD20 monoclonal antibodies according to the present invention can be also used as an active ingredient of therapeutic agents for the treatment of malignant tumors of B-cells and immune diseases involving B-cells. Therefore, the present invention, in further embodiments, provides therapeutic agents for the treatment of malignant tumors of B-cells and immune diseases involving B-cells, in which the therapeutic agents contain an antibody of the series 1782 as an active ingredient. Methods by and amounts at which the therapeutic agents are administered can be easily determined by those skilled in the art. As diseases involving B-cells are exemplified the above-described diseases.

The present invention will be described in more detail hereinafter with reference to the Examples. However, the invention is not limited to the Examples.

### Example 1

### (1) Preparation of Immunization Agents for Sensitizing Mice

SB and Raji cells, which are of B-cell strains expressing CD20, were cultured in vitro.

Separately, DNA coding for the entire CD20 molecule (Multiple Choice cDNA human spleen, Origene Technologies, Inc., 6 Taft Court, Suite 100, Rockville, Md. 20850) was cloned using specific primers hCD20-S-GK-Not: aatgcggccgccaccatgacaacacccagaaattc (SEQ ID No: 25) and hCD20-E-Xba: gctctagattaaggagagctgtcattttc (SEQ ID No: 26).

The DNA was inserted into a high expression vector for mammalian cells, pNOW, (FIG. 1) and a constructed vector was used to transform CHO cells. Recombinant CHO cells (CD20/CHO cells) displaying high levels of expression of CD20 on the cell surface were identified using FACS analysis. Staining was performed using an FITC-labeled anti-CD20 monoclonal antibody and cells were selected as high expression cell, when they gave five or more times the fluorescence intensity of SB cells.

### (2) Preparation of Immunogens

SB or Raji cells were cultured using RPMI 1640 medium supplemented with 10% FCS. CD20/CHO cells were cultured using CHO-S-SFM II medium (Gibco, Cat. No. 12052-098) supplemented with 800 μg/ml of G418. These cultured mediums were centrifuged for 5 minutes at 1,100 rpm, the cells were suspended in Dulbecco's PBS(−) and centrifuged again. This washing step was repeated once again, and physiological saline was added to the cells to prepare a suspension (cell number: 1-3×10⁷/ml), which was used for immunization.

### (3) Immunization

These immunogen preparations were administered intraperitoneally to female Balb/c mice of 7- to 11-week old. After administration of either of the SB or CD20/CHO cells was repeated two to three times at intervals of various days, the final immunization was carried out using cells of a different type (CD20/CHO or Raji cells). The number of cells administered was 1-3×10⁷ cells per mouse for each of these immunizations.

The combinations of immunogens used are shown in Table 1.

### (4) Cell Fusion

Three days after the final immunization, spleen cells were prepared from two mice and fused with mouse myeloma cells (NS−1) in the presence of PEG-1500 according to the method described in Oi, V. T. and L. A. Herzenberg, 1980, in: Selected Methods in Cellular Immunology, eds.: B. Mishell and S. M. Shiigi (Freeman and Co., San Francisco, Calif.) p. 351.

### (5) Primary and Secondary Screening

Cell ELISA was performed using 96-well plates having CD20/CHO or CHO cells (parent strain) attached thereto to select wells where an antibody reacting specifically to CD20 was produced. 96-well plates with the same CD20/CHO cells attached thereto were subjected competitive reaction with rituximab (C2B8) to select antibodies (wells) which reacted to an epitope similar to that of C2B8.

The results of these screenings are shown in Table 1.

### (6) Cell ELISA

CD20/CHO or CHO cells (parent strain) attached to a Poly-L-Lysine coated 96-well plate (Asahi Technoglass Corporation, Cat. No. 11-023-018) were used for Cell ELISA. 150 μl of blocking buffer (PBS solution with 0.2% gelatin, 0.5% BSA) was placed into each well and the plate was allowed to stand at 37° C. for one hour. The plate was washed five times using an aqueous solution of 150 nM NaCl, 0.05% Tween 20, and then a 100-μl sample (a diluted solution of a cultured supernatant) was placed into each well. The primary reaction was conducted at 37° C. for one hour. After washing, 100 μl of a diluted solution of a labeled antibody (HRP-labeled anti-mouse IgG (H+L) rabbit antibody (Jackson Lab., Code No. 315-035-003) or HRP-labeled anti-mouse IgG (Fcγ) rabbit antibody (Jackson Lab., Code No. 315-035-008)) was placed into each well and a secondary reaction was conducted at 37° C. for one hour. The same blocking solution was used in the preparation of the reaction solution for the primary and the secondary reactions. After washing, 100 μl of a color developing solution (OPD) was placed into each well and 30 minutes later, 50 μl of 4N H₂SO₄ was added to stop the reaction. The absorbance was measured at 492 nm.

### (7) Competitive Reaction in Cell ELISA

Mixed solutions of a sample (diluted solution of a cultured supernatant) and a chimerized antibody (10 to 40 ng/ml) were prepared.

After blocking reaction was carried out as in the above-described Cell ELISA, 100 μl of each of the mixed solutions was placed into each well and the primary reaction was performed at 37° C. for one hour. After washing, 100 μl of a diluted solution of a labeled antibody (HRP-labeled anti-human IgG (H+L) rabbit antibody (Jackson Lab., Code No. 309-035-082)) was placed into each well and a secondary reaction was conducted at 37° C. for one hour. After washing, 100 μl of a color developing solution (OPD) was placed into each well and 30 minutes later, 50 μl of 4N H₂SO₄ was added to stop the reaction. The absorbance was measured at 492 nm.

Since the labeled antibody reacts only with the chimerized antibody, any competition between the antibody in the sample added in the primary reaction and the chimerized antibody results in reduction in the measured value.

### (8) Cloning

A limiting dilution method was used. After culturing cells seeded on 96-well plates, Cell ELISA was performed on cultured supernatants of wells having a single colony, thereby to select clones producing a specific antibody.

### (9) Preparation of Purified Antibodies

Clones producing a specific antibody were cultured in RPMI 1640 medium supplemented with 10% FCS. When the cell density reached approximately 5×10⁵ cells/ml, the medium was replaced by serum-free culture medium ASF-104N (Ajinomoto). Two to four days later, the cultured medium was centrifuged to collect the cultured supernatant. A protein G column was used for purification of the antibody. A solution of the eluted monoclonal antibody was dialyzed using 150 mM NaCl. The dialyzed solution was filter sterilized through a 0.2 μm filter to obtain an antibody sample to be tested (anti-human CD20 mouse monoclonal antibody).

TABLE 1

| | | | | Primary, | | |
|---|---|---|---|---|---|---|
| | | | | Secondary Screening | | |
| | Immunizing Method | | | Specificity against CD20 | | |
| | Initial and Booster | | | on CD20/CHO cells | | |
| Cell | Immunization, | | Number of | Number of | | Number of |
| Fusion | Number of | Final | immunized | selected wells | | measured |
| Series | Immunizations | Immunization | mice | A | B | wells |
| 1K18 | SB cell, 3 times | Raji cell | 2 | 7 | 2 | 576 |
| 1K20 | Raji cell, 3 times | SB cell | 2 | 7 | 0 | 576 |
| 1K14 | SB cell, 2 times | CD20/CHO | 1 | 20 | 9 | 576 |
| | | cell | | | | |
| | SB cell, 3 times | CD20/CHO | 1 | | | |
| | | cell | | | | |
| 1K17 | CD20/CHO cell | Raji cell | 1 | 21 | >10 | 576 |
| | 2 times | | | | | |
| | CD20/CHO cell | Raji cell | 1 | | | |
| | 3 times | | | | | |

Number of selected wells-A: wells where an antibody reacting with CD20/CHO cells and not with CHO cells was produce.
Number of selected wells-B: of the wells selected in A, wells where an antibody undergoing a competitive reaction with the reference antibody (C2B8) was produce.

The amino acid sequences of the L-chain V-region (SEQ ID Nos: 1 to 8) and the H-chain V-region (SEQ ID Nos: 9 to 16) of monoclonal antibodies produced by 8 representative clones are shown below.

The sequence of the H-chain V-region of 1K0924 (SEQ ID No: 11):

QVQLQQPGAELVKPGASVKMSCKASGYTFTSYNIHWVKQTPGQGLEWIGA
IYPGNGDTSYNQKFKGKATLTSDKSSSTAYMQLSSLTSEDSAVYYCARMS
TMITGFDYWGQGTTLTVSS

The sequence of the H-chain V-region of 1K1228 (SEQ ID No: 16):

QVQLQQPGAELVKPGASVKVSCKASGFTFTSYNLHWVKQTPGQGLVWIGA
IYPGNGDTSYNQKFRGKATLTADISSSTAYMQLSSLTSEDSAVYYCARYY
YGYDAMDYWGQGTSVTVSS

The sequence of the H-chain V-region of 1K1422 (SEQ ID No: 9):

QVQLQQPGAELVKPGASVKMSCRASGYTFTNYNMHWIKQTPGQGLEWIGA
IYPGSGDTSYNRKFKGKATLTADTSSSTAYMQFSSLTSADSAVYYCARFT
YYYGGTYGAMDYWGQGTSVTVSL

The sequence of the H-chain V-region of 1K1791 (SEQ ID No: 10):

QIQLVQSGPELKKPGETVKISCKASGYTFTNFGVNWVKQAPGKGLKWMGW
INTYTGEPSYADDFKGRFAFSLEASANTAYLQINNLKNDDMSTYFCTRRT
NYYGTSYYYAMDYWGQGTSVTVSS

The sequence of the H-chain V-region of 1K1712 (SEQ ID No: 12):

QVQLQQPGAELVKPGASVKMSCKASGFTFTSYNLHWVKQTPGQGLEWIGA
IYPGSGDTSYNQQFKGKATLTADKSSNTAYMQLNSLTSEDSAVYCCARSA
MISTGNWYFDYWGQGTTLTVSS

The sequence of the H-chain V-region of 1K1402 (SEQ ID No: 13):

QVQLQQPGAELVKPGASVKMSCKASGFTFTSYNMHWVKQTPGQGLEWIGG
IYPGNGDTSYNQKFKGKATLTADKSSSTAYMQLSSLTSEDSAVYYCARFY
YYGSMGAMDYWGQGTSVTVSS

The sequence of the H-chain V-region of 1K1736 (SEQ ID No: 14):

QVQLQQPGAELVKPGASVKMSCKASGYTFTTYNLHWVKQTPGQGLEWIGG
IYPGNGDTSYNQKFKVKATLTADKSSNTAYMQLSSLTSEDSAVYYCARWI
YYGNYEGTLDYWGQGTSVTVSS

The sequence of the H-chain V-region of 1K1782 (SEQ ID No: 15):

QVQLQQSGAELAKPGASVKMSCKASGYTFTSYWMHWVKQRPGQGLEWIGY
ITPSTGYTDYNKKFKDKATLTADRSSSTAYMHLSSLTSEDSAVYYCARSG
PYFDVWGAGTTVTVSS

The sequence of the H-chain V-region of 1K0924 (SEQ ID No: 3):

QIVLSQSPAILSASPGEKVTMTCRASSSVSYMHWYQQRPGSSPKPWIYAT
SNLASGVPARFSGSGSGTSYYFTISRVEAEDAATYYCQQWNSNPPTHGGG
TKLEIK

The sequence of the H-chain V-region of 1K1228 (SEQ ID No: 8):

EIILTQSPTTMAASPGEKITITCSASSSISSYYLRWYQQKPGFSPKVLIY
RTSNLASGVPARFSGSGSGTSYSLTIGTMEAEDVATYYCQQGNTVPLTFG
SGTKLEIK

The sequence of the L-chain V-region of 1K1422 (SEQ ID No: 1):

QIVLTQSPPIMSASLGEEITLTCSASSRVSYMLWYQQKSGTSPKLLIYST
SNLASGVPSRFSGSGSGTFYSLTISSVEAEDAADYYCHQWTSNPCTFGGG
TKLEIK

The sequence of the L-chain V-region of 1K1791 (SEQ ID No: 2):

STVMTQTPKFLLVSAGDRVTITCKASQSVSNDVAWYQQKPGQSPKVLIYF
ASNRYTGVPDRFTGSGYGTDFTFTINTVQAEDLAVYFCQQDYSSPLTFGA
GTKLELK

The sequence of the L-chain V-region of 1K1712 (SEQ ID No: 4):

QIVLSQSPAILSASPGEKVTMTCRASSSVSYMDWYQQKPGSSPKPWIYAT
SNLASGVPARFSGSGSGTSYSLTISRVEAEDTATYYCQQWTFNPPTFGSG
TKLEIK

The sequence of the L-chain V-region of 1K1402 (SEQ ID No: 5):

QIVLSQSPAILSASPGEKVTMTCRASSSVSYMHWYQQKPGSSPKPWIYAT
SNLASGVPARFSGSGSGTSYSLTITRVEAEDAATYYCQQWTFNPPTFGAG
TKLELK

The sequence of the L-chain V-region of 1K1736 (SEQ ID No: 6):

QIVLSQSPAILSSSPGEKVTMTCRASSSVSYMLWYQQKPGSSPEPWIYAT
SNLASGVPARFSGGGSGTSYSLTISRVEAEDAATYYCQQWTFNPPTFGGG
TKLEIK

The sequence of the L-chain V-region of 1K1782 (SEQ ID No: 7):

DILLTQSPAILFVSPGERVSLSCRASQNIGTSIHWYQQRTNGSPRLLIKY
ASESFSGIPSRFSGSGSGTDFTLSINSVESEDIADYYCQQSNSWPFTFGS
GTKLEIK

### Example 2

For some of the resulting clones, the base sequences of their monoclonal antibody genes in the variable region were determined. In addition, antibody binding affinities were measured and biological property tests were performed for the monoclonals produced by them, as described below.

### (1) Measurement of Binding Affinity

Floating Raji cells derived from human B-cell, which express the target antigen on the cell surface, and floating Jurkat cells derived from human T-cell, which do not express CD20 antigen were used. Cells of both types were cultured in a CO₂ incubator (SANYO MCO-175M) at 37° C. and at a CO₂ concentration of 5% using RPMI 1640 medium (NACALAI TESQUE, Inc., Cat. No. 30264-85, Lot L4K2844) supplemented with 10% fetal calf serum (FCS) (BIOLOGICAL IND., Cat. No. 04-001-1A, Lot 815242; preheated at 56° C. for 30 minutes inactivation of the complement components). The cells were maintained by subculturing twice a week.

Measurements of the number of cells were made using a Burker-Turk hemacytometer (Erma, inc., Cat. No. 03-303-1).

Cultured media of confluent cells three to four days after subculturing were centrifuged for three minutes at 3,000 rpm at room temperature using a multi-rack refrigerated centrifuge LX-120 (TONY Co., Ltd.). The supernatant was removed and the cells were collected. The rotation speed and time used in this step were selected so that the number of cells remained unchanged after the repetition of centrifugal separation and supernatant removing. For removing the culture medium and FCS remaining on the cell surface (washing), the collected cells were suspended in Dulbecco's Phosphate Buffered Saline (−) (free of Ca and Mg, PBS(−), Wako, Cat. No. 191-01665, Na₂HPO₄: Wako, Cat. No. 197-02865, Lot ASF2635, KCl: Wako, Cat. No. 163-0334T, Lot CEQ7122, KH₂PO₄: Wako, Cat. No. 169-0425, Lot ELG7616)) and centrifuged twice for 3 minutes at 3,000 rpm to remove the supernatant. The cells after washing were suspended in a solution of 1% BSA (Wako, Cat. No. 013-07492 Lot PKH3483) in PBS and adjusted to a cell density of 5×10⁶ cells/ml.

As a primary antibody, an antibody to be tested or a positive control antibody (2B8) was dispensed into 1.5-ml tubes (BM Equipment Co., Ltd., BM ring-lock tubes, Cat. No. BM-15) in the respective amounts of 15, 30, 50, 75, 100, 125, 150, and 200 ng (1.5 to 5 μl). At the same time, four tubes with no added antibody were prepared. Three samples were prepared for each antibody to be tested. To each of the samples was added 100 μl (5×10⁵ cells) of a suspension in a PBS solution of 1% BSA (Wako, Cat. No. 013-07492, Lot PKH3483) and mixed, and reacted with shaking at room temperature for one hour.

After reacting, the reaction solutions were centrifuged at 3,000 rpm for 3 minutes at room temperature using a high-speed refrigerated microcentrifuge MX-100 (TONY). After collecting the cells, the cells were suspended in 200 μl of PBS and centrifuged at 3,000 rpm for three minutes to remove the supernatant. These procedures were repeated twice to remove any unreacted primary antibody remaining on the cell surface.

An FITC-labeled anti-mouse IgG (H&L) secondary antibody [GOAT Anti-mouse IgG (H&L) Fluorescein conjugated, affinity purified Secondary antibody, Chemicon, Cat. No. AP124F, Lot 24021014] was added in excess (500 ng) with respect to the primary antibody binding to cells, followed by 100 μl of a 1% BSA-PBS solution (500 ng/100 μl). The mixture was shaken for one hour at room temperature while being shielded from the light, and the primary antibody binding to cells was detected. After the reaction, the mixture was centrifuged at 3,000 rpm for three minutes to collect the cells. In order to remove any unreacted FITC-labeled anti mouse IgG (H&L) antibody remaining on the cell surface, the cells were suspended in 200 μl of PBS and centrifuged at 3,000 rpm for three minutes to remove the supernatant. These procedures were repeated twice.

The cells thus collected were suspended in 100 μl of PBS and transferred to flat-bottomed 96-well plates (Sumitomo Bakelite Co., Ltd., ELISA PLATE, Cat. No. 8496F). The intensity of fluorescence from the secondary antibody was measured using a Typhoon 9210 Image Analyzer (Amersham Bioscience) under the following detection conditions: Fluorescence mode, 600 V, 526SP/green(532 nm), Focus: +3 mm above the bottom face. Controls for constructing a standard curve were prepared using PBS solutions of 100 μl, to which 0, 12.5, 25, 50, 75, 100, 125, and 150 ng of the FITC-labeled secondary antibody were added.

After the detection, images were digitized using an image analysis software, Image Quant (Amersham Bioscience), and analyzed using Excel (Microsoft). Background values resulting from the plate, PBS solution, and FITC-labeled secondary antibody binding non-specifically to cells were determined by measuring reactions only between the cells and the FITC-labeled secondary antibody, and the average value for the four points were subtracted from the value of fluorescence intensity for each sample. In this way, the amount of fluorescence from the FITC-labeled secondary antibody binding to cells was obtained. A standard curve was constructed by measuring amounts of fluorescence at various concentrations of the FITC-labeled secondary antibody used as control, and the amount of secondary antibody binding to cells (number of moles or weight) was calculated. The amount of primary antibody binding cells was calculated assuming that each of the primary antibodies and the FITC-labeled secondary antibody react at a ratio of 1:2. The amount of free primary antibody was determined by subtracting the binding amount from the added amount. When the antibody concentration was converted into molar concentration, the molecular weights of the monoclonal antibodies were taken to be 150,000.

It was observed that the binding reaction became saturated with increasing amounts of primary antibody added and the intensity of fluorescence reached a constant value. Scatchard analysis was used to calculate the number of antigens on the cell surface and the dissociation constant (Kd value) (see Scatchard, G., Ann. N.Y. Acad. Sci., 51: 660-672, 1949; New Cultured Cell Experimental Methods in Molecular Biology Research, Yodosha Co., Ltd., Jikken Igaku separate volume, BioManual UP Series Revised 2^{nd} Edition, pages 212 to 217). The values used were the average of three values for the respective samples.

The measurement results for monoclonal antibodies produced by 8 representative clones and a positive control antibody (2B8) are shown below in Table 3.

### (2) Biological Property Tests

### (a) Apoptosis Induction Test

The ability of test antibodies to induce apoptosis was measured using flow cytometry (Annexin V/PI staining). A positive control (2B8) and a negative control (Anti-CD3 monoclonal antibody (BD PharMingen) were used. The test was performed using a MEBCYTO Apoptosis Kit (MBL, Cat. No. 4700, Lot. 20).

Raji cells were centrifuged, and suspended in fresh RPMI 1640 medium (Sigma, Cat. No. R8758, Lot. 44K2416) supplemented with 10% (inactivated) FBS (ICN, Cat. No. 2916754, Lot. 8005C) and placed into each well of 12-well plates in a volume of 1 ml at a density of 5×10⁵ cells/ml. Twelve wells per antibody were used for testing, and an antibody was added to the respective wells to give a final concentration of 2 or 4 μg/ml (3 wells×2 concentrations×2 time points, 12 wells in total). On days 1 and 2 after the culture was started, the cultured media containing about 2×10⁵ cells were removed and after centrifugation, the cells were washed once in PBS. Then, the cells were suspended in 85 μl of binding buffer. After mixing well with 10 μl of Annexin V-FITC and 5 μl of PI, the mixture was allowed to react for 15 minutes at room temperature while being shielded from the light. Measurements were made using flow cytometry (FACS Calibur, Becton Dickinson) and analyzed with CellQuest (Becton Dickinson).

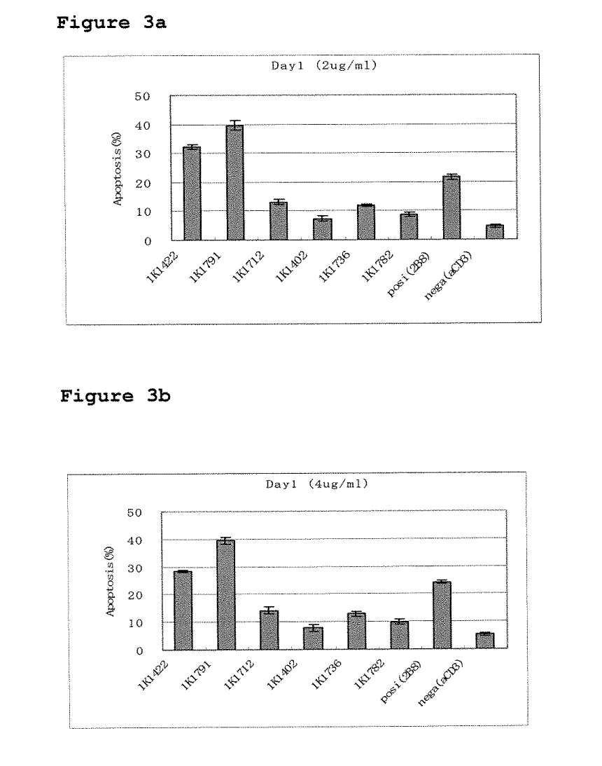

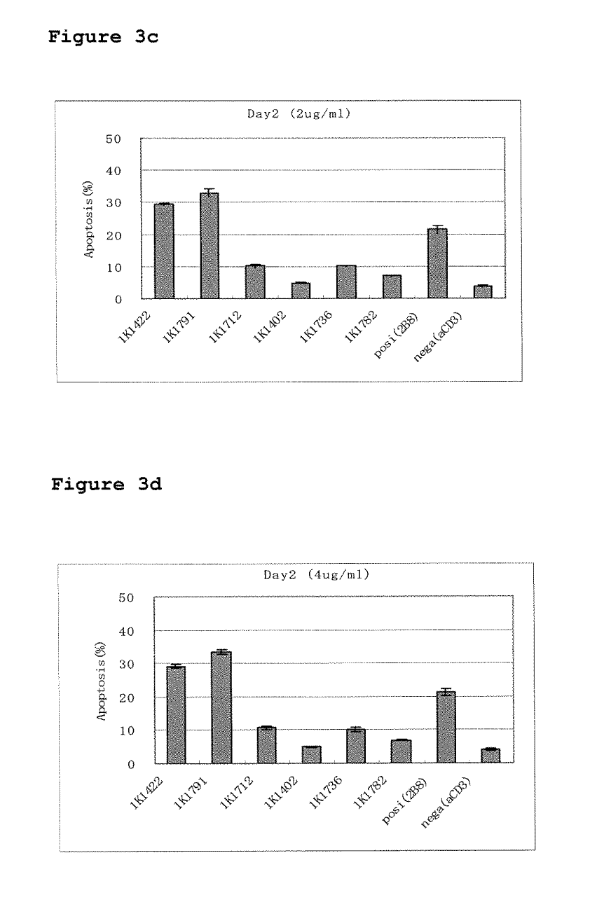

The measurement results for monoclonal antibodies produced by 6 representative clones, a positive control antibody (2B8), and a negative control antibody (Anti-CD3) are shown below in FIG. 3a to FIG. 3d. Although 2B8 is in general believed to have a high ability to induce apoptosis, the monoclonal antibodies produced by the clone 1k1791 obtained from cell fusion of the series 1K17 (immunization with CD20/CHO cells and Raji cells) and by the clone 1k1422 obtained from cell fusion of the series 1K14 (immunization with SB cells and CD20/CHO cells) were found to display high abilities to induce apoptosis when compared with 2B8.

### (b) Cell Growth Inhibition Test

A suspension of Raji cells with a cell concentration 5×10⁴ cells/ml was prepared in RPMI 1640 supplemented with 10% FCS, added at a volume of 100 μl per well into 96-well plates, and cultured. After 24 hours, an antibody solution was added at a volume of 50 μl per well to give an antibody concentration of 1 μl/ml, and culturing was continued. At 72 hours after addition of the antibody, 10 μl/well of a color developing dye, Cell Counting Kit-8 (Dojindo Laboratories, Cat. No. 343-07623, Lot. SG076), was added, culturing was continued for another 4 hours, and then absorption was measured at 492 nm.

The absorbance measurement results for monoclonal antibodies from the six clones as described above, a positive control antibody (2B8), and a negative control are shown below in Table 2, and their properties are shown in Table 3.

### Cell Growth Inhibition Test

TABLE 2

| Clone name | Absorption (492 nm) |
|---|---|
| 1K1422 | 1.775 |
| 1K1791 | 1.794 |
| 1K1712 | 2.326 |
| 1K1402 | 2.540 |
| 1K1736 | 2.239 |
| 1K1782 | 2.603 |
| Positive control (2B8) | 1.759 |
| Negative control | 2.607 |

### Properties of Monoclonal Antibodies

TABLE 3

| | | Binding | | Cell Growth |
|---|---|---|---|---|
| | | affinity | Ability | Inhibiting |
| | | Kd value | to Induce | Effect (in |
| Clone name | Isotype | (nM) | Apoptosis | Vitro) |
| 1K1422 | IgG1, κ | 3.39 | 130 | present |
| 1K1791 | IgG1, κ | 1.70 | 160 | present |
| 1K0924 | IgG2b, κ | 1.35 | 60 | present |
| 1K1712 | IgG2a, κ | 0.84 | 50 | — |
| 1K1402 | IgG1, κ | 0.78 | 30 | — |
| 1K1736 | IgG2b, κ | 0.54 | 50 | — |
| 1K1782 | IgG1, κ | 0.40 | 30 | — |
| 1K1228 | IgG1, κ | 0.26 | 30 | — |
| Positive control (2B8) | IgG1, κ | 6.79 | 100 | present |

### (c) Antibody-Dependent Cell-Mediated Cytotoxicity (ADCC)

In this series of experiments, measurements were made of whether effector cells could become activated through the Fc region of an anti-CD20 chimerized antibody, thereby to lyse cells of lymphoma cell lines.

Four types of cells, Raji, WiL2-NS, SU-DHL4, and RC-K8 cells derived from human B-cell, were cultured and maintained in an incubator at 37° C. and at a CO₂ concentration of 5% in RPMI 1640 medium supplemented with 10% inactivated FCS (culture medium).

In these experiments, cells of each type were washed in RPMI 1640 containing 10% FCS and reacted with calcein for 15 minutes at 37° C., whereby the calcein was allowed to be incorporated into viable cells, and after that, the number of cells was adjusted to 4×10⁵ cells/ml. Since calcein is retained only in cells having the cell membrane kept in normal conditions, it can be used to stain only viable cells. Rituximab (C2B8), which is an anti-CD20 chimerized antibody, and 6 types of chimerized antibodies (1k0924, 1k1402, 1k1422, 1k1712, 1k1736, and 1k1791) were adjusted to be at 20, 4, and 0.8 μg/ml using RPMI 1640 containing 10% FCS. Effector cells were adjusted to be at 5×10⁶, 1×10⁶, and 0.2×10⁶ cells/ml after effector cells were obtained by taking blood from healthy subjects and immediately layering the blood onto a Ficoll, followed by centrifugation to collect a lymphocyte fraction.

In a total of 100 μl, 25 μl of a cell suspension having an adjusted concentration, 25 μl of each of the anti-CD20 chimerized antibody solutions (with the respective final concentrations of 5, 1, and 0.2 μg/ml), and 50 μl of effector cell for each of the antibody concentrations (with the respective E:T ratios of 25:1, 5:1 and 1:1) were mixed well in 96-well plates and reacted for 4 hours in an incubator at 37° C. and at a CO₂ concentration of 5%. Three samples were prepared as follows: a first sample in which the antibody solution and the effector cells were replaced with RPMI 1640 containing 10% FCS, in order to calculate the spontaneous lysis of cells; a second sample in which the antibody solution was replaced with RPMI 1640 containing 10% FCS, which was for calculating the antibody-independent activity of only the effector cells; and a third sample in which the antibody solution was replaced with 20% Triton X-100, which was for calculating the maximum lysis.

After the reaction, if cells have been lyzed, then the cell membrane has been broken and the calcein has been released out of the cell. Based on this, the fluorescence of calcein released into the reaction solution was quenched using a Quencher and the amount of fluorescence was then measured using a fluorescence analyzer.

After the detection, images were digitized using an image analysis software (Amersham Bioscience), and the lysis rate for each sample was calculated using the equation below.

Lysis rate(%)=((Spontaneous lysis)−(Sample))/((Spontaneous lysis)−(Maximum lysis))×100 Equation 1

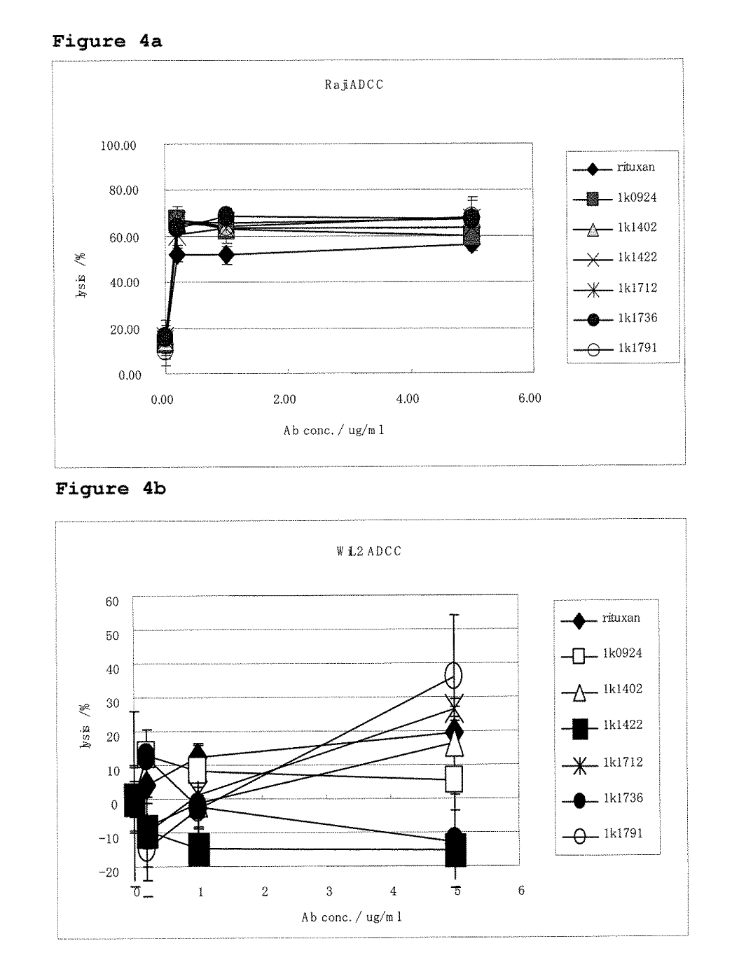

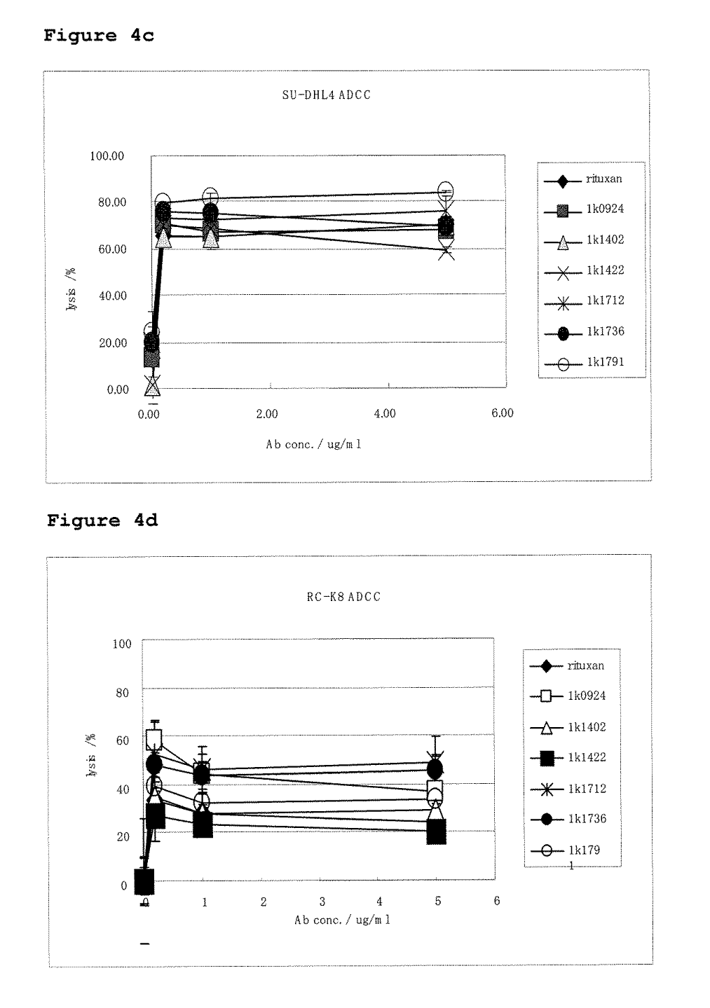

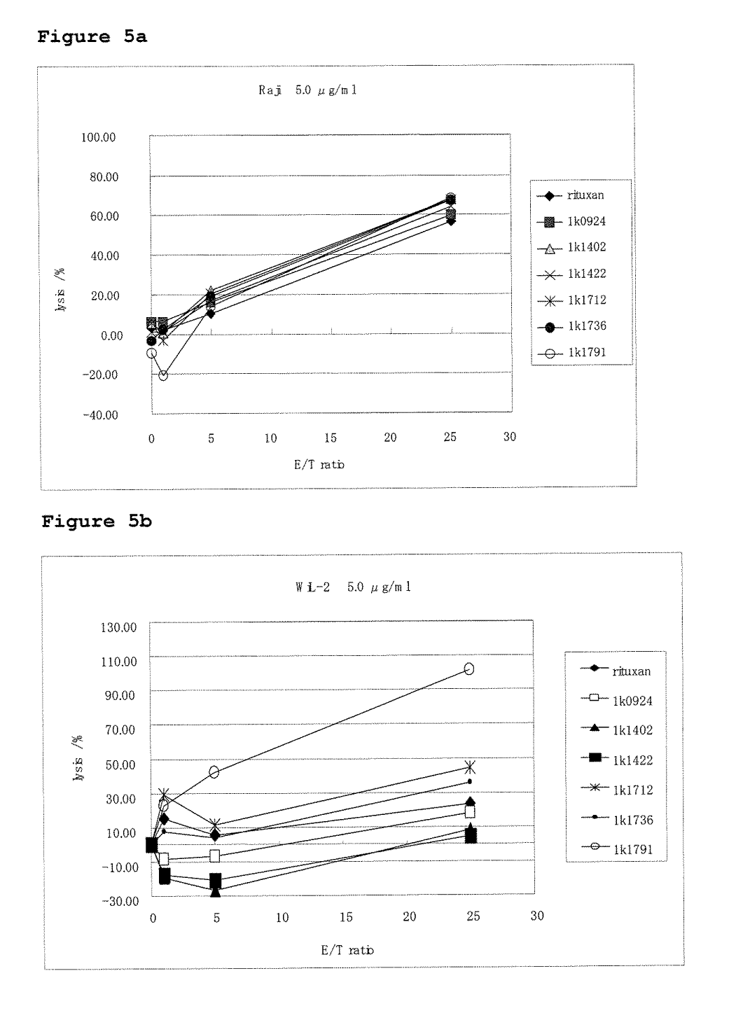

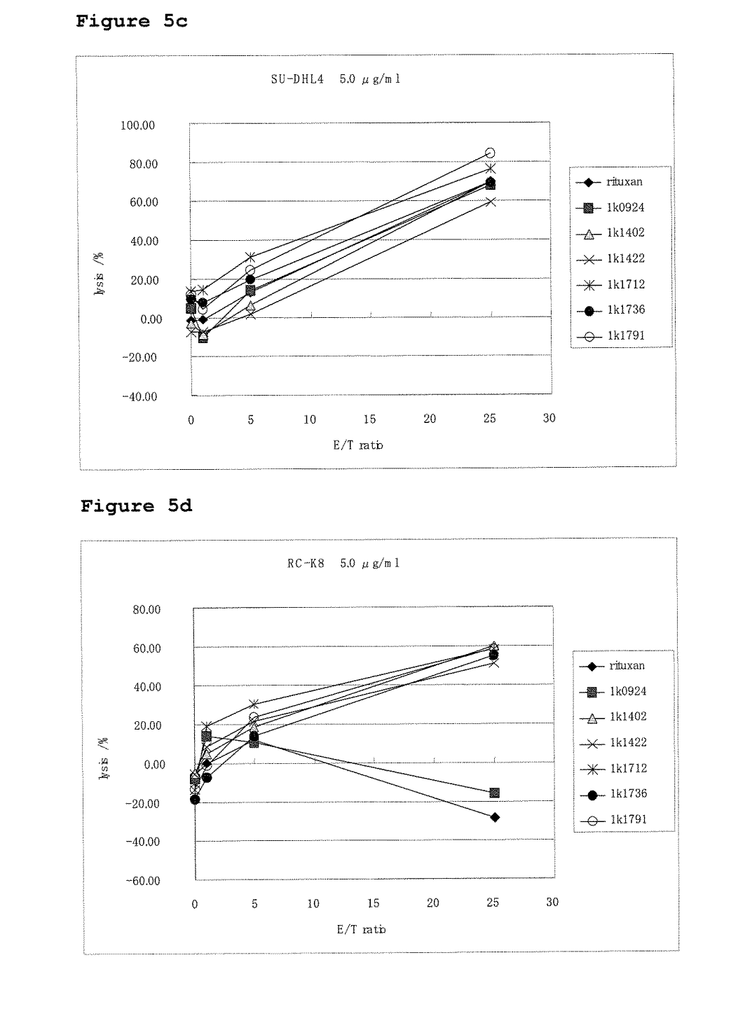

FIG. 4a to FIG. 4d show the relationship between antibody concentration and cytotoxic activity for each of the cell types, when the ratio of the number of effector cells, such as NK cells, and the number of target cells (E:T ratio) was 25:1. FIG. 5a to FIG. 5d show the relationship between the E:T ratio and cytotoxic activity for each of the cell types, when the antibody concentration was 5 μg/ml.

As shown in FIG. 4a to FIG. 4d, all of the cell types shows cytotoxic activity by adding the respective antibodies, under conditions where the E:T ratio was 25:1. In other words, the antibodies participate in cytotoxicity. Furthermore, cell strains other than WiL2-NS display, already at an antibody concentration of 0.2 μg/ml, activities equivalent to those at antibody concentrations of 1 and 5 μg/ml (which become saturated at 0.2 μg/ml) and the activity becomes constant after reaching a maximum, suggesting that these effects are provided in amounts of antibody which are less than the amount of antibody required for complement-dependent cytotoxic activity.

As shown in FIG. 5a to FIG. 5d, it would be recognized that from the effect of E:T ratio on cytotoxic activity at an antibody concentration of 5 μg/ml, the cytotoxic activity increases depending on the E:T ratio concentration. From this, it can be concluded that the cytotoxicity is caused by the action of effector cells.

### (d) Complement Dependent Cytotoxicity (CDC)

In this series of experiments, measurements were made of whether the anti-CD20 chimerized antibodies could lyse cells of lymphoma cell lines in the presence of serum containing complements.

Four types of cells, Raji, WiL2-NS, SU-DHL4, and RC-K8 cells derived from human B-cell, were cultured and maintained in an incubator at 37° C. and at a CO₂ concentration of 5% in RPMI 1640 supplemented with 10% inactivated FCS (culture medium).

In these experiments, cells of each type were washed in RPMI 1640 containing 10% FCS and the number of cells was adjusted to 2-3×10⁶ cells/ml. C2B8 (rituximab), which is an anti-CD20 chimerized antibody, and 6 types of chimerized antibodies (1k0924, 1k1402, 1k1422, 1k1712, 1k1736, and 1k1791) were adjusted to be at 20, 4, and 0.8 μg/ml using RPMI 1640 containing 10% FCS.

In a total of 100 μl, 55 μl of a cell suspension having an adjusted concentration, 25 μl of each of the anti-CD20 chimerized antibody solutions (with the respective final concentrations of 5, 1, and 0.2 μg/ml), and 20 μl of pooled serum taken from five healthy subjects or an inactivated serum thereof were mixed well using a vortex mixer and reacted for 2 hours in an incubator at 37° C. and at a CO₂ concentration of 5%. Samples in which 25 μl of the antibody solution was replaced with RPMI 1640 containing 10% FCS were prepared as samples for calculation of backgrounds.

After the reaction, dead cells were stained using PI (propidium iodide) and analyzed by FACS (Becton Dickinson). As numerical values, populations obtained of dead cells were directly used, from which the values of background and of the sample to which the inactivated serum was added were subtracted.

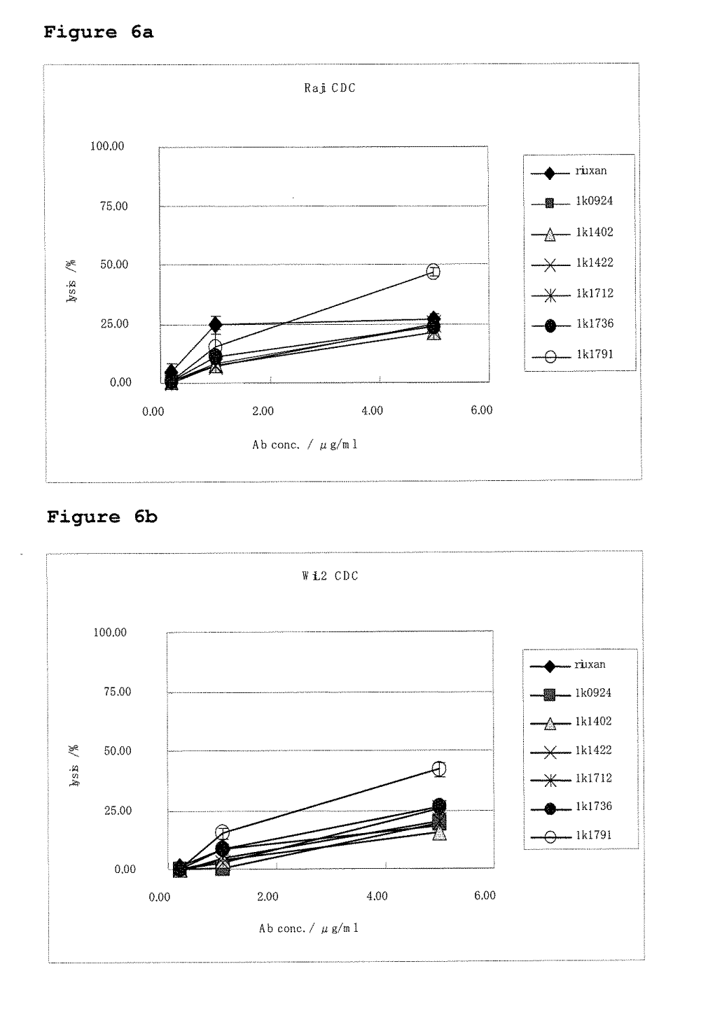

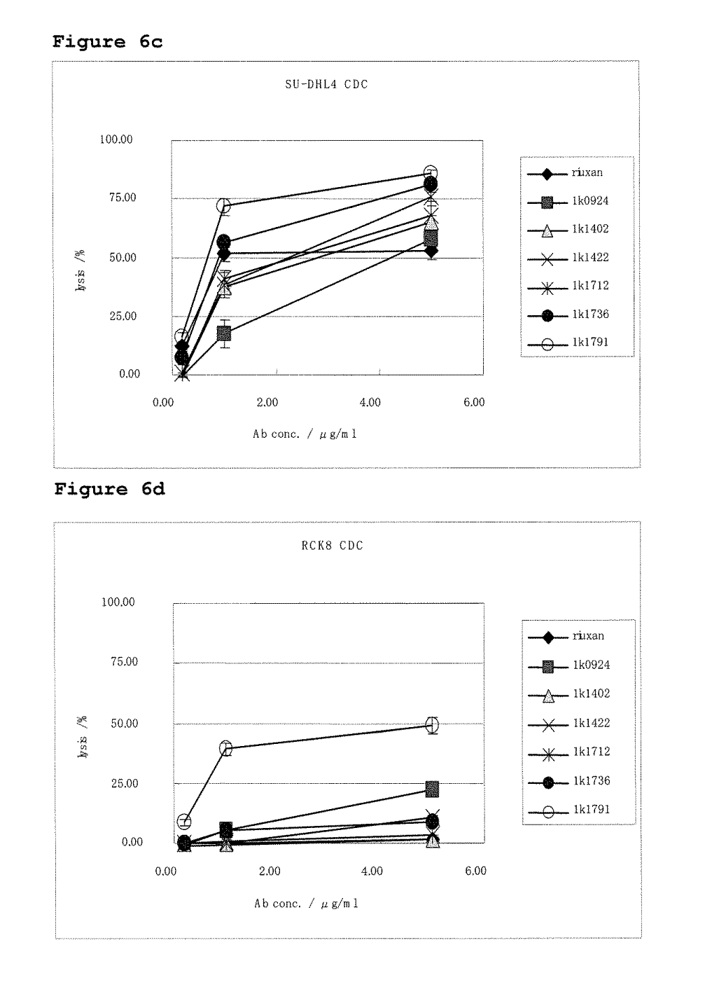

FIG. 6a to FIG. 6d show the relationship between antibody concentration and cytotoxic activity for each cell type.

As shown in FIG. 6a to FIG. 6d, all the six types of antibodies show activity in Raji, WiL2-NS, and SU-DHL4. Furthermore, the concentration dependency can be confirmed, and when compared at a concentration of 5 μg/ml, 1k1791 shows particularly high activities relative to the other antibodies, followed by 1k1736, 1k1422, and 1k1712 showing high activities. At this concentration, it can be recognized that in Raji and WiL2-NS cells, 1k1791 induces cytotoxicity at levels approaching about two times those induced by the other antibodies, which also display activities equal to or greater than rituximab.

RC-K8 cells on which rituximab is considered to have no effect, on the other hand, were found to remarkably show differences between the respective antibodies. Rituximab, 1k1402, and 1k1712 show little or no activity against RC-K8 cells. In contrast, 1k1791 displays very high activity and shows a cytotoxic activity of approximately 50% at a concentration of 5 μg/ml. Subsequently to 1k1791, 1k0924 shows a cytotoxic activity of 25% or so, and 1k1422 and 1k1736 show a cytotoxic activity of 10% or so.

From the above, it can be confirmed that the 6 types of chimerized antibodies which were used as the subject of these tests display CDC activities equal to or stronger than rituximab.

### Example 3

### (1) Measurement of Binding Affinity

Raji cells derived from human B-cell were cultured in a CO₂ incubator at 37° C. and at a CO₂ concentration of 5% using RPMI 1640 medium supplemented with 10% inactivated FCS. The cells were maintained by subculturing twice a week.

Cell cultures three to four days after subculturing (containing approximately 1×10⁶ cells/ml) were centrifuged for five minutes at 1,000 rpm at room temperature to collect the cells. The cells were suspended in PBS(−) and centrifuged for five minutes at 1,000 rpm to remove the supernatant. These procedures were repeated twice for washing the cells.

The reaction with a primary antibody was performed by mixing the respective anti-CD20 antibodies (mouse antibody 2B8 as positive control antibody, chimerized antibodies, and humanized antibody C2B8) with Raji cells and subjecting the mixtures to reaction at room temperature for one hour. In the reaction, the respective anti-CD20 antibodies had twelve final concentrations of 1.33, 2.67, 4.00, 5.33, 6.67, 8.00, 9.33, 10.67, 12.00, 13.33, 14.67, and 16.00 nM, the number of cells was 5×10⁶ cells, and the reaction solution used a 1% BSA/PBS solution, which was dispensed into 1.5-ml tubes to a final volume of 100 μl. Three samples for each antibody to be tested were prepared, and as samples for calculation of backgrounds, four tubes to which no antibody was added were also prepared.

After the reaction, the reaction mixtures were centrifuged at room temperature for three minutes at 3,000 rpm to remove any unreacted primary antibody and to collect the cells.

A solution of an FITC-labeled secondary antibody prepared at a concentration of 5 μg/ml in a 1% BSA/PBS solution was added at a volume of 100 μl into the tubes, so that the FITC-labeled secondary antibody was in excess with respect to the primary antibody binding to cells, and the mixture, after suspending, was reacted for one hour at room temperature while being shielded from the light.

The FITC-labeled secondary antibodies used were GOAT Anti Mouse IgG (H&L)-FITC for the mouse antibody, and GOAT F(ab′) 2F fragment [sic] Anti Human IgG (Fcγ)-FITC for the chimerized and humanized antibodies.

After the reaction, the mixtures were centrifuged at 3,000 rpm for three minutes at room temperature. Unreacted FITC-labeled secondary antibody was removed and the cells were collected. The cells were washed by suspending them in 200 μl of PBS and centrifuging them again.

The cells were suspended in 100 μl of PBS and transferred into flat-bottomed 96-well plates. The intensity of fluorescence of the secondary antibody was measured using a Typhoon 9210 Image Analyzer (Amersham Bioscience).

After the detection, images were digitized using an image analysis software, Image Quant (Amersham Bioscience) and analyzed using Excel (Microsoft). The average value for the same tested antibody was calculated and the average value obtained from the samples for background calculation (in which only the cells and the FITC-labeled secondary antibody were reacted) was subtracted from the value for each of the tested antibodies, thereby to eliminate the background value resulting from the plate, PBS solution, and FITC-labeled secondary antibody binding non-specifically to cells. Additionally, a standard curve was constructed by measuring amounts of the fluorescence from solutions containing only the FITC-labeled secondary antibody in amounts of 0, 12.5, 25, 50, 75, 100, 125, and 150 ng per 100 μl. This standard curve was used to determine the number of moles of the secondary antibody binding to cells. Assuming that each of the primary antibodies and the FITC-labeled secondary antibody react at a ratio of 1:5, the amount of primary antibody binding to cells was calculated. The amount of free primary antibody was calculated by subtracting the binding amount from the added amount. These values were used for Scatchard analysis, thereby to calculate the dissociation constant Kd.

The results are shown below in Table 4.

### (2) Apoptosis Induction Test

Initial apoptosis was detected using a Annexin V-FITC apoptosis kit under two reaction conditions, a first condition where measurements were made of apoptosis which an anti-CD20 antibody alone induced against cells derived from human B-cell stains (non-crosslinking condition), and a second condition where measurements were made of apoptosis which was induced by adding a secondary antibody recognizing the Fc region of an anti-CD20 antibody (crosslinking condition).

Four types of cells, Raji, WIL2-NS, SU-DHL4, and RC-K8 cells derived from human B-cell, were cultured in a CO₂ incubator at 37° C. and at a CO₂ concentration of 5% in RPMI 1640 supplemented with 10% inactivated FCS (culture medium). The cells were maintained by subculturing twice a week.

Three to four days after subculturing, cell cultures (approximately 1×10⁶ cells/ml) were centrifuged for five minutes at 1,000 rpm at room temperature to collect the cells.

The respective anti-CD20 antibodies (mouse antibody 2B8 as positive control antibody, chimerized antibodies, and humanized antibody C2B8; Anti-CD2 monoclonal antibody as negative control) were mixed with the cells suspended in fresh culture medium and the mixtures were reacted for 1.5 hours in an incubator at 37° C. and at a CO₂ concentration of 5%. In the reaction, the respective anti-CD20 antibodies had three final concentrations of 0.2, 1, and 5 μg/ml, the cell density was 1×10⁶ cells/ml, and the reaction solution used culture medium, which was placed in a volume of 250 μl into 1.5-ml tubes and subjected to reaction. Three samples were prepared for each antibody to be tested.

After the reaction, the mixtures were centrifuged at 1,200 rpm for three minutes at room temperature to remove unreacted antibody and to collect the cells.

Under the non-crosslinking condition, 250 μl of fresh culture medium was added to the cells. Under the crosslinking condition, 250 μl of a secondary antibody recognizing the Fc region of the anti-CD20 antibody, which was in an amount of five times the amount of the CD20 antibody, was added to the cells. After mixing, the mixtures were reacted for another three hours in an incubator at 37° C. and at a CO₂ concentration of 5%. The secondary antibodies used were Goat Anti mouse IgG, Fcγ Fragment for the mouse antibody, and Goat Anti human IgG, Fcγ Fragment specific, for the chimerized and humanized antibodies.

After the reaction, the mixtures were centrifuged at 3,000 rpm for three minutes at room temperature to remove unreacted secondary antibody and to collect the cells. The cells were washed by suspending them in 250 μl of PBS and centrifuging them again.

The reagent for detection used a MEBCYTO apoptosis kit-Annexin V-FITC, PI- (MBL, Cat. No. 4700, Lot. 21). After the cells were suspended in 85 μl of Binding buffer, μl of Annexin V-FITC and 5 μl of propidium iodide (PI) (a final concentration of 0.5 mg/ml) were added and mixed well, and the mixture was reacted for 15 minutes at room temperature while being shielded from the light.

20,000 cells in a total count were measured using flow cytometry (EPICS ALTRA: BECKMAN COULTER) and analyzed (Expo32: BECKMAN COULTER).

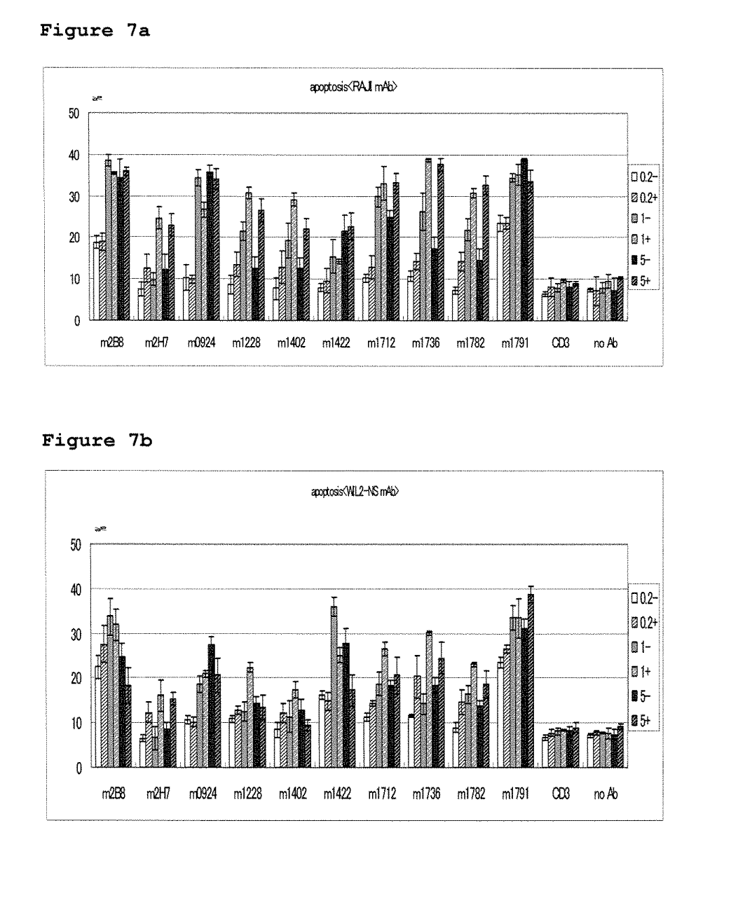

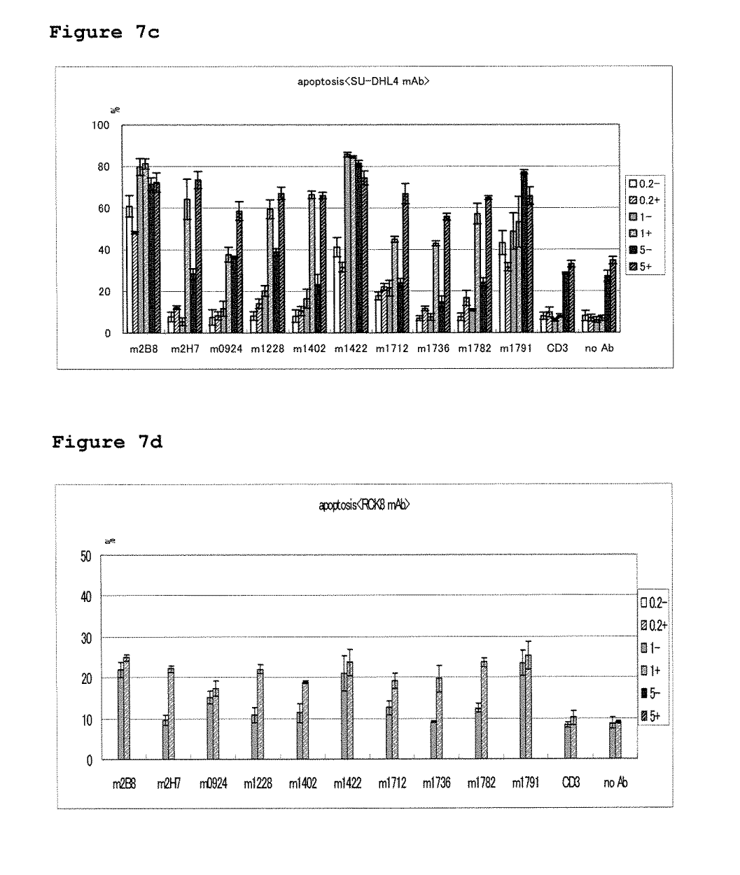

The results are shown in FIG. 7a to FIG. 9d.

### (3) Preparation of Humanized Antibody Producing Strains

### (a) DNA Synthesis

DNAs with codons optimized for CHO cells, based on the amino acid sequences set forth in SEQ ID Nos: 17 to 24, were designed and synthesized in routine procedures.

### (b) Preparation of Constructs

Sixteen types of expression constructs for humanized 1K1791 were constructed using pNOW as an expression vector. Expression constructs for humanized 1K1791 were made. pNOW-aa1791 kg1, pNOW-af1791 kg1, pNOW-as1791 kg1, pNOW-av1791 kg1, pNOW-fa1791 kg1, pNOW-ff1791 kg1, pNOW-fs1791 kg1, pNOW-fv1791 kg1, pNOW-sa1791 kg1, pNOW-sf1791 kg1, pNOW-ss1791 kg1, pNOW-sv1791 kg1, pNOW-va1791 kg1, pNOW-vf1791 kg1, pNOW-vs1791 kg1, pNOW-vv1791 kg1.

### (c) Transfection and Selection Using Chemical Reagents

The respective expression constructs for humanized 1K1791 were introduced into CHO DG44cdB cells using a transfection reagent. 1×10⁶ CHO DG44cdB cells into which each of the genes was introduced were suspended in 100 ml of selective medium, seeded on five 96-well plates (200 μl/well), and cultured for 3 to 4 weeks at 37° C. under an atmosphere of 5% carbon dioxide gas.

Transfection reagent: Qiagen, Effectene Transfection Reagent, Cat. No 301427.

Selective Medium: IS CHO-CD w/Hydrolysate/4 mM GlutaMAX/0.8 mg/ml G418.

### (d) Selection of High Expression Cell Strains

1) Supernatants were recovered from wells where colonies were present and the amount of antibody produced was measured using a Dot Blot assay.
2) Clones displaying high amounts of antibody produced were transferred into 24-well plates and after culturing for approximately 5 days, the supernatants were recovered and the amount of antibody produced was measured using Sandwich ELISA
3) Two clones displaying high levels of expression were selected and transferred into T75 flasks.

### (e) Small Scale Culturing

Two clones selected for each of the 16 types of constructs were cultured in T75 flasks containing 30 ml of the selective medium.

### (4) Culturing of Humanized Antibody Producing Strains and Purification

A antibody producing cell strain (genetically recombinant CHO-DG44 cells) was cultured in Hydrolysate-containing IS CHO-CD/with Hydrolysate medium (Irvine Scientific, Cat. No. 91119) supplemented with 4 mM GlutaMax (Invitrogen, Cat 35050-061) and 200 μg/ml of G418 (Sigma, Cat. No. A1720-5G) in a CO₂ incubator at 37° C. and at a CO₂ concentration of 5%. The cells were maintained by subculturing twice a week.

Cell culture about two weeks after subculturing was started was centrifuged at 3,500 rpm for five minutes at room temperature to collect the supernatant, which in turn was filtered using a 0.45-μm syringe filter and equilibrated with 50 mM Tris-HCl, pH 7.0.

The supernatant was loaded onto a Hi Trap Protein A HP column (GE Healthcare, Cat No. 17-0402-01), and then washed using 50 mM Tris-HCl, pH 7.0. Elutions were obtained using 0.1 M citric acid, pH 4.0. Fractions of 400 μl were collected and neutralized with 40 μl, corresponding to 10/1 [sic] of the fraction volume, of 1 M Tris-HCl pH 9.0. Dialysis was performed against PBS of 100-times volume using Slyde-A Lyzer 10K Dialysis Cassetes (PIERCE, Cat. No. 66453) twice for a period of 2.5 hours each, then once for a period of 15 to 18 hours. The purified sample was filtered using a 0.22-μm syringe filter, followed by concentration using a VIVA SPIN 50,000 MWCO PES (VIVASCIENCE, Cat. No. VS0231). The antibody concentration was calculated from the A280 value using a BECKMAN COULTER DU530.

The antibody producing strains used were CHO cells hz1791-fv10, hz179′-ff34, hz1791-sf43, and hz1791-ss32, which were deposited under Accession Nos. FERM BP-10543, FERM BP-10544 FERM BP-10545, and FERM BP-10546, respectively, with the International Patent Organism Depositary, National Institute of Advanced Industrial Science and Technology, Tsukuba Central 6, 1-1-1 Higashi, Tsukuba, Ibaraki, Japan, on 1 March in the 18th year of Heisei (2006), under the provisions of the Budapest Treaty.

The amino acid sequences of the H-chain V-region and the L-chain V-region of humanized anti-CD20 monoclonal antibodies obtained from these cell strains (SEQ ID Nos: 17 to 24) are described hereinafter (the underlined amino acids differ from those of the corresponding mouse antibodies).

Sequences for Humanized 1k1791:

The sequence of the L-chain V-region (SEQ ID No: 20):

Ven 1791: STVMTQSPDSLAVSLGERVTINC KASQSVSNDVA
WYQQKPGQSPKVLIY FASNRYT
GVPDRFSGSGYGTDFTFTISSVQAEDVAVYFC QQDYSSPLT
FGAGTKLELK

The sequence of the H-chain V-region (SEQ ID No: 24):

Ven 1791: QIQLVQSGPELKKPGASVKISCKASGYTFT NFGVN
WVKQAPGKGLKWMG WINTYTGEPSYADDFKG RFAFSLDASVSTAY
LQISSLKAEDTSTYFCTR RTNYYGTSYYYAMDY WGQGTTVTVSS

The sequence of the L-chain V-region (SEQ ID No: 17):

abb 1791: STVMTQSPDSLAVSLGERATINC KSSQSVSNDVA
WYQQKPGQSPKVLIY FASNRYS
GVPDRFSGSGYGTDFTLTISSLQAEDVAVYFC QQDYSSPLT
FGAGTKLEIK

The sequence of the H-chain V-region (SEQ ID No: 21):

abb 1791: QIQLVQSGSELKKPGASVKVSCKASGYTFT NFGVN
WVRQAPGKGLEWMG WINTYTGEPSYAQGFTG RFVFSLDASVSTAY
LQISSLKAEDTATYFCTR RTNYYGTSYYYAMDY WGQGTTVTVSS

The sequence of the L-chain V-region (SEQ ID No: 19):

sdr 1791: STVMTQSPDSLAVSLGERATINC KSSQSNSNDVA
WYQQKPGQSPKVLIY FASNRYS
GVPDRFSGSGYGTDFTLTISSLQAEDVAVYFC QQDYSSPLT
FGAGTKLEIK

The sequence of the H-chain V-region (SEQ ID No: 23):

sdr 1791 QIQLVQSGSELKKPGASVKVSCKASGYTFT NFGVN
WVRQAPGKGLKWMG WINTYTGEPSYAQGFTG RFAFSLDASVSTAY
LQISSLKAEDTATYFCTR RTNYYGTSYYYAMDY WGQGTTVTVSS

The sequence of the L-chain V-region (SEQ ID No: 18):

fra 1791: STVMTQSPSFLSASVGDRVTITC KASQSVSNDVA
WYQQKPGQSPKVLIY FASNRYT
GVPDRFSGSGYGTDFTLTISSLQAEDVAVYFC QQDYSSPLT
FGAGTKLEIK

The sequence of the H-chain V-region (SEQ ID No: 22):

fra 1791: QIQLVQSGSELKKPGASVKVSCKASGYTFT NFGVN
WVKQAPGKGLKWMG WINTYTGEPSYADDFKG RFAFSLDASASTAY
LQISSLKAEDMATYFCTR RTNYYGTSYYYAMDY WGQGTTVTVSS

In this series of experiments, experiments were performed with respect to two clones derived from each of a total of 16 sequence-combinations resulting from combining four types which were AbbL, FraL, SdrL, and VenL, and four types which were AbbH, FraH, SdrH, and VenH. From the above results, these clones can be classified into 4 groups as shown in Table 5, based on their Nd values. One clone was selected from each of the groups and used in further experiments.

In the specification, for example, when clone fv of humanized antibody 1791 is referred to, it means an antibody clone in which the L-chain of fra1791 (the L-chain set forth in SEQ ID No: 18) and the H-chain of Ven1791 (the H-chain set forth in SEQ ID No: 24) are combined (underlining is done for explanation). This similarly applies to other clones.

Control: c2B8
Group I: Kd=20 nM<

### Group II: Kd=10 to 20 nM

### Group III: Kd=8 to 10 nM

### Group IV: Kd=<8 nM

### Binding Dissociation Constants of Humanized Antibodies

TABLE 4

| Hz1791 clone No. | Kd (nM) | C2B8 Kd (nM) |
|---|---|---|
| Aa008 | 11.68 | 6.61 |
| Aa012 | 9.96 | 4.68 |
| Fa007 | 11.34 | 5.63 |
| Fa008 | 8.83 | 4.91 |
| Sa023 | 14.66 | 6.13 |
| Va016 | 13.09 | 6.67 |
| Va024 | 7.50 | 6.47 |
| Af021 | 6.84 | 5.56 |
| Af025 | 8.67 | 4.29 |
| Ff019 | 8.50 | 5.70 |
| Ff034 | 7.81 | 4.00 |
| Sf043 | 11.60 | 4.34 |
| Sf056 | 13.79 | 6.01 |
| Vf029 | 7.78 | 3.32 |
| Vf031 | 7.79 | 4.54 |
| As001 | 11.89 | 5.74 |
| As002 | 11.47 | 8.7 |
| Fs007 | 6.33 | 5.63 |
| Fs024 | 11.09 | 3.97 |
| Ss020 | 24.39 | 4.10 |
| Ss032 | 21.79 | 7.25 |
| Vs006 | 8.8 | 4.23 |
| Vs011 | 11.9 | 6.09 |
| Av004 | 10.71 | 5.76 |
| Av006 | 9.17 | 4.86 |
| Fv010 | 7.17 | 4.39 |
| Fv028 | 7.25 | 4.74 |
| Sv015 | 14.31 | 6.30 |
| Sv020 | 10.85 | 5.36 |
| Vv018 | 7.14 | 5.04 |
| Vv023 | 7.01 | 4.86 |

### Binding Dissociation Constants of Humanized Antibodies

TABLE 5

| Group | Hz1791 clone | Kd (nM) |
|---|---|---|
| I (20 nM <) | ss | 23.09 |
| II (10 to 20 nM) | sa | 14.66 |
| | sf | 12.70 |
| | sv | 12.58 |
| | as | 11.68 |
| | aa | 10.82 |
| | vs | 10.35 |
| | va | 10.30 |
| | fa | 10.09 |
| III (8 to 10 nM) | av | 9.94 |
| | fs | 8.71 |
| | ff | 8.15 |
| IV (<8 nM) | vf | 7.78 |
| | af | 7.76 |
| | fv | 7.21 |
| | vv | 7.07 |
| Control | c2B8 | 5.35 ± 1.13 |

The results of these experiments for apoptosis using 8 types of mouse antibodies are shown in FIG. 7a to FIG. 7d. The inventors were able to divide the 8 types of mouse antibodies and known CD20 antibodies, 2B8 and 2H7, into two groups with respect to all the four types of cells (some exceptions were not included).

Group A: m0924, m1422, m1791, m2B8
Group B: m1228, m1402 m1712, m1782, m2H7
(However, m0924 was included in Group B with respect to SU-DHL4 cells).

In other words, Group A includes anti-CD20 antibodies which display sufficient capabilities to induce apoptosis by themselves and have approximately the same level of apoptosis inducing capability even under the crosslinking condition with a secondary antibody, while Group B includes anti-CD20 antibodies which display insufficient capabilities to induce apoptosis by themselves and have an greatly increased capabilities to induce apoptosis under the crosslinking condition. Furthermore, the antibodies belonging to Group A display affinities comparable to that of 2B8, while the antibodies belonging to Group B display higher affinities than that of 2B8. Consequently, it can be inferred that the antibodies of Group A are more suitable for pharmaceutical use since they do not require the presence of a secondary antibody to induce apoptosis and are capable of inducing apoptosis by themselves.

Turning to the type of cells, the results indicate that RAJI, WIL2-NS, RCK8 cells had ratios of apoptosis, at highest, in the range of 30 to 40%, whereas SU-DHL4 cells displayed maximum ratios of not less than 80% in some cases.

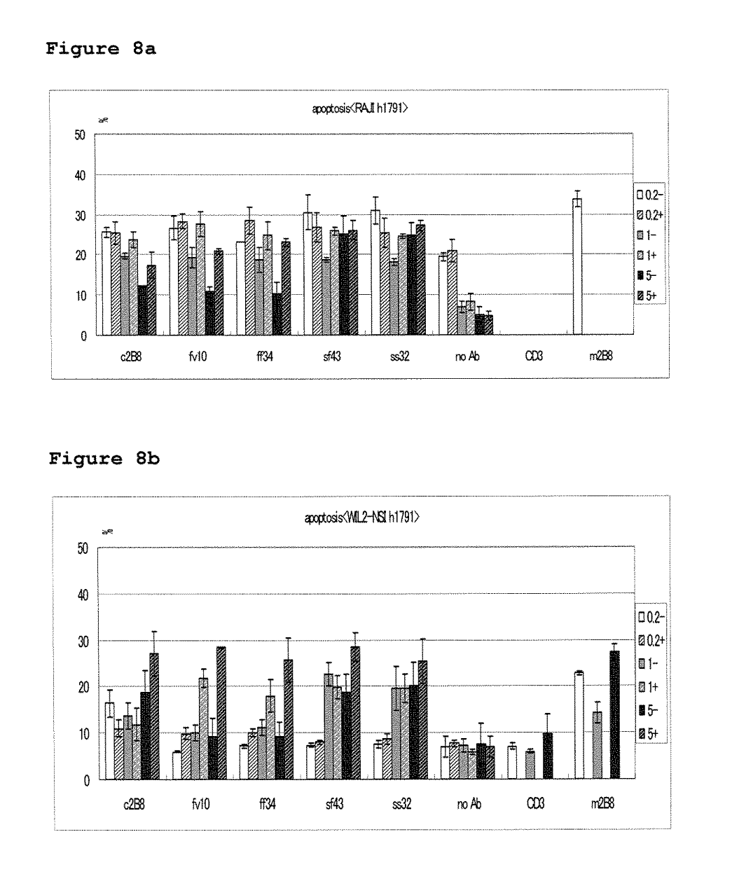

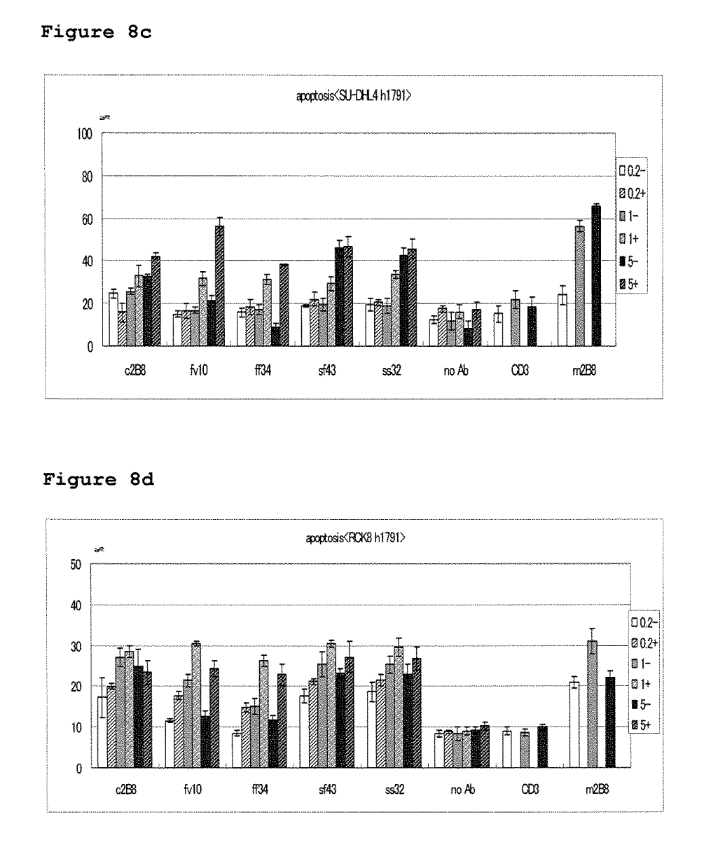

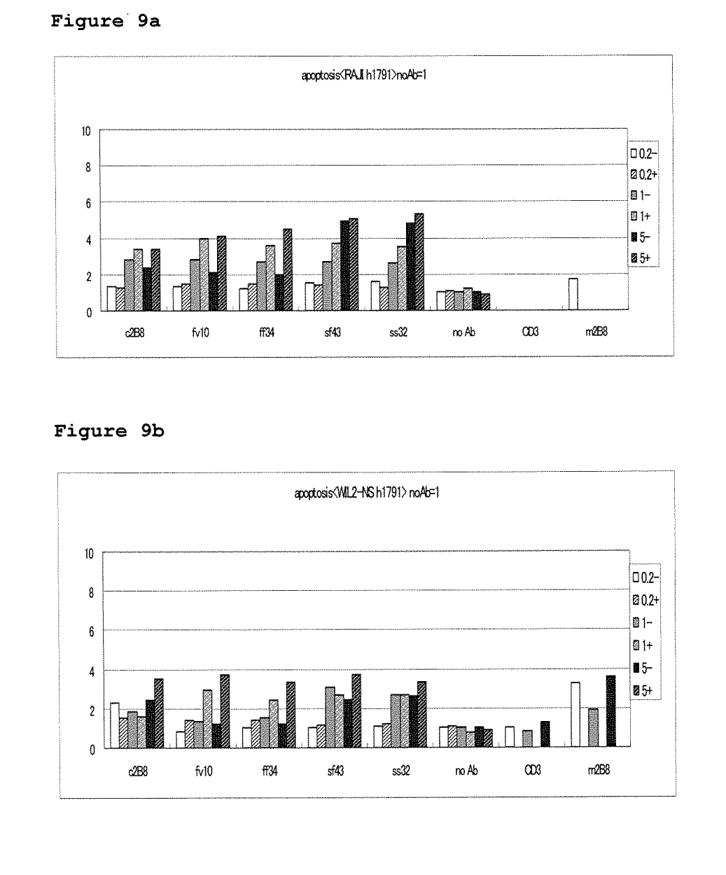

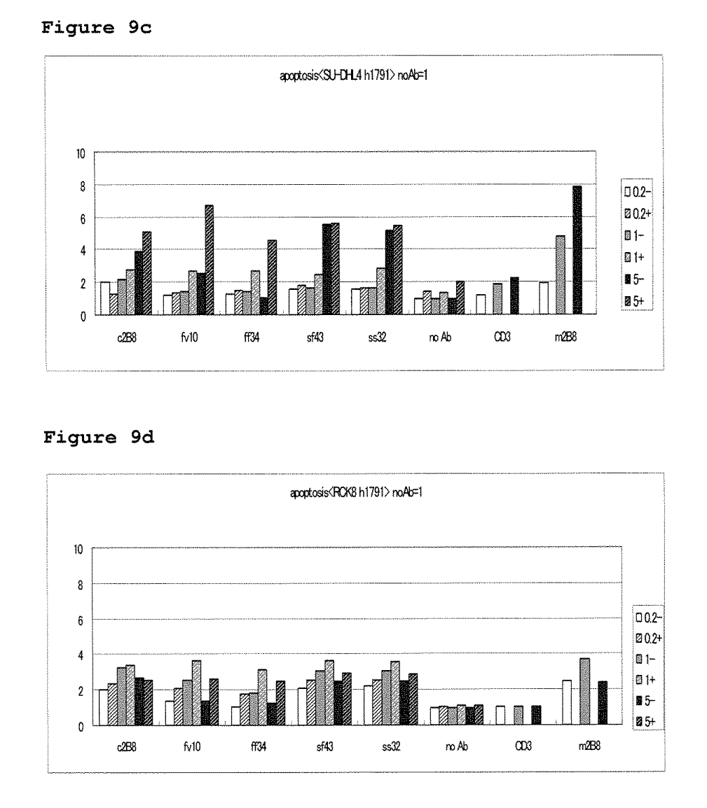

The results for the four types of humanized antibodies are shown in FIG. 8a to FIG. 9d.

Similarly to the mouse antibodies, four clones of humanized antibody 1791 were classified into two groups with respect to all the four types of cells.

Group A: fv10, ff34
Group B: sf43, ss32, c2B8

Antibodies fv10 and ff34 of Group A, which display comparable affinities to that of C2B8, have almost the same apoptosis activity as that of C2B8 under the non-crosslinking condition and clearly increased activities under the crosslinking condition. Although antibodies sf and ss of Group B display greater dissociation constants and weaker affinities than the antibodies of Group A, these antibodies alone display slightly stronger apoptosis activities than C2B8.

As concerns the capability of the anti-CD20 antibodies to induce apoptosis against B-cells, when they display sufficient capabilities of inducing apoptosis by themselves, the ratio of apoptosis is substantially the same under the crosslinking condition. However, when they do not display a sufficient capability of inducing apoptosis by themselves, due to the type of antibodies, unsaturated conditions, or the like, it is likely that apoptosis activity will increase under the crosslinking condition.

FIG. 9a to FIG. 9d are graphs showing early apoptosis (%) wherein the ratio of early apoptosis under conditions of “no Ab” (no added antibody) on the day of the experiments is set to 1.

Apoptosis activities of four clones of humanized antibody 1791, clones fv, ff, sf, and ss, against Raji, SU-DHL4, WiL-2, and RCK8 cells are summarized in Table 6.

### Summary of Apoptosis Activities of Humanized Antibody-1791 Clones

TABLE 6

| | Affinity | Apoptosis (5 ug/ml) | | | | | | | |
|---|---|---|---|---|---|---|---|---|---|
| | (average) | RAJI | | Wil2-NS | | DHL4 | | RCK8 | |
| | Raji | | XL | | XL | | XL | | XL |
| Antibody | Kd (nM) | m2B8 = 100 | w/wo | m2B8 = 100 | w/wo | m2B8 = 100 | w/wo | m2B8 = 100 | w/wo |
| c2B8 | 5.35 | 100 | 1.4 | 100 | 1.4 | 100 | 1.3 | 100 | 0.9 |
| fv | 7.21 | 88 | 1.9 | 49 | 3.1 | 65 | 2.7 | 50 | 1.9 |
| ff | 8.15 | 84 | 2.2 | 49 | 2.8 | 96 | 1.2 | 47 | 1.9 |
| sf | 12.70 | 205 | 1.0 | 100 | 1.5 | 143 | 1.0 | 93 | 1.2 |
| ss | 23.09 | 202 | 1.1 | 108 | 1.3 | 133 | 1.1 | 92 | 1.2 |
| no Ab | | 42 | 0.9 | 41 | 0.9 | 26 | 2.0 | 37 | 1.1 |

CDC activities were measured for the clones fv, ff, sf, and ss, of humanized antibody 1791 using Raji, SU-DHL4, WiL-2, and RCK8 cells. The respective results are shown in Table 7. Rituximab (c2B8) and the clone 1791 (c1791) were included as controls.

CDC Activities of Humanized Antibodies according to the Present Invention

TABLE 7

| | CDC (10 ug/ml) | | | |
|---|---|---|---|---|
| | RAJI | WIL2NS | SUDHL4 | RC-K8 |
| Antibody | c2B8 = 100 | c2B8 = 100 | c2B8 = 100 | c2B8 = 100 |
| c2B8 | 100 | 100 | 100 | 3 |
| fv | 99 | 172 | 120 | 498 |
| ff | 98 | 203 | 121 | 530 |
| sf | 78 | 295 | 111 | 713 |
| ss | 73 | 120 | 61 | 108 |
| c1791 | 99 | 529 | 119 | 100 |

The results in Table 7 show that the clones ff and fv displayed CDC activities which were equal to or stronger than that of rituximab. Furthermore, sf showed extremely strong CDC activities with respect to WiL2 and RCK8 cells.

Experiments were also conducted to investigate the relationship between antibody concentration and CDC activity. The antibodies used were purified to a higher degree of purification than that of the antibodies prepared as described in Example 2. Purification was carried out in the procedures described below.

A antibody producing cell strain (genetically recombinant CHO-DG44 cells) was cultured in Hydrolysate-containing IS CHO-CD/w medium (Irvine Scientific, Cat. No. 91119) supplemented with 4 mM GlutaMax (Invitrogen, Cat. 35050-061) and 200 μg/ml of G418 (Sigma, Cat. No. A1720-5G) in a CO₂ incubator at 37° C. and at a CO₂ concentration of 5%. The cells were maintained by subculturing twice a week. Cell culture approximately two weeks after subculturing was centrifuged at 3,500 rpm for five minutes at room temperature to collect the supernatant, which in turn was filtered using a 0.45-μm syringe filter and equilibrated with 50 mM Tris-HCl, pH 7.0. The supernatant was loaded onto a Hi Trap Protein A HP column (GE Healthcare, Cat. No. 17-0402-01), and then washed using 50 mM Tris-HCl, pH 7.0. Elutions were obtained using 0.1 M citric acid, pH 4.0. Fractions of 400 μl were collected and neutralized with 40 μl, corresponding 10/1 [sic] of the fraction volume, of 1 M Tris-HCl, pH 9.0. Dialysis was performed against PBS of 100-times volume using Slyde-A-Lyzer 10K Dialysis Cassetes (PIERCE, Cat. No. 66453) twice for a period of 2.5 hours each, then once for a period of 15 to 18 hours.

The dialyzed sample was concentrated using a VIVASPIN 50,000 MWCO PES (VIVASCIENCE, Cat. No. VS0231). The sample was loaded onto a HiLoad 16/60 superdex 200 prep grade column (GE Healthcare Cat No. 17-1069-01) equilibrated with PBS. The purified sample was filtered using a 0.22-μm syringe filter and concentrated using a VIVASPIN 50,000 MWCO PES (VIVASCIENCE, Cat. No. VS0231). The antibody concentration was calculated from the A280 value using a BECKMAN COULTER DU530.

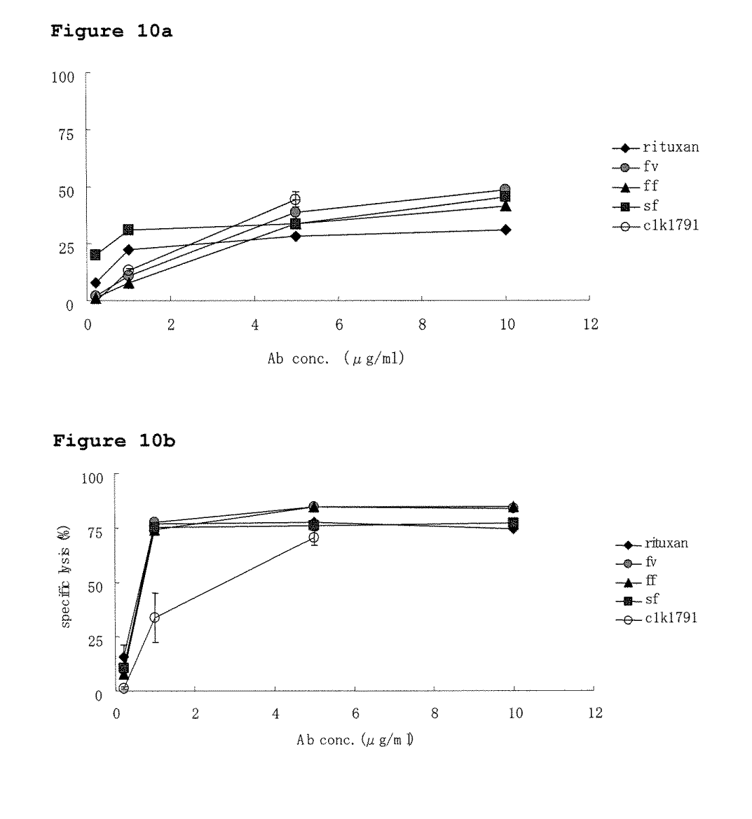

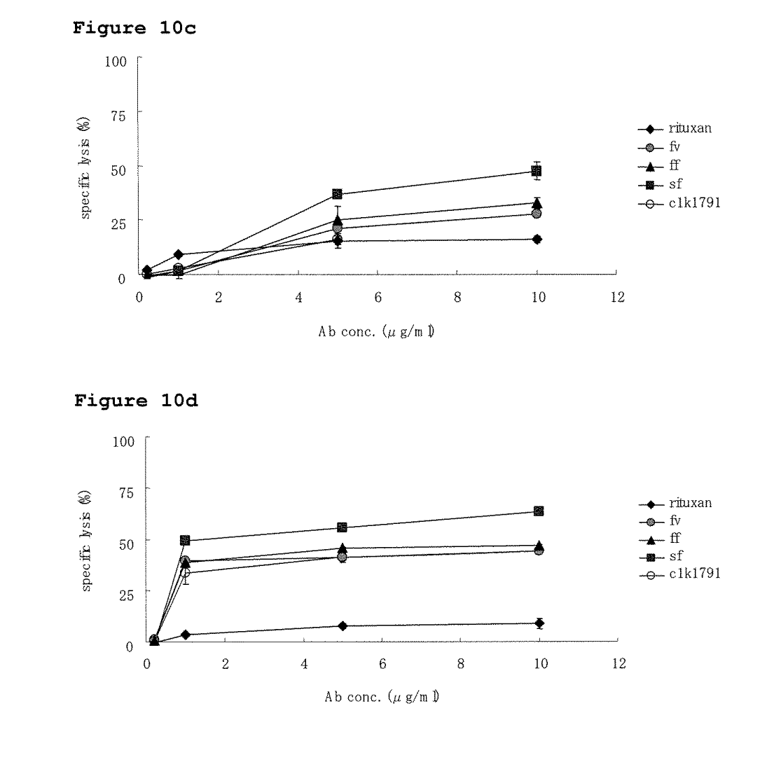

FIG. 10a to FIG. 10d show the relationship between concentration and CDC activity of the humanized antibodies fv, ff, sf, and ss, and the chimerized antibody c1k179, for Raji, SU-DHL4, WiL-2, and RCK8 cells. In all the experiment systems, CDC activities of the humanized antibodies fv, ff, sf, and ss at a concentration of 5 μg/ml or more were equal to or greater than that of Rituxan (C2B8). In particular, although Rituxan displayed little CDC activity against RC-K8 cells, the four clones of humanized antibody 1791 according to the present invention displayed CDC activities against RC-K8 cells at least about 4 times higher than Rituxan.

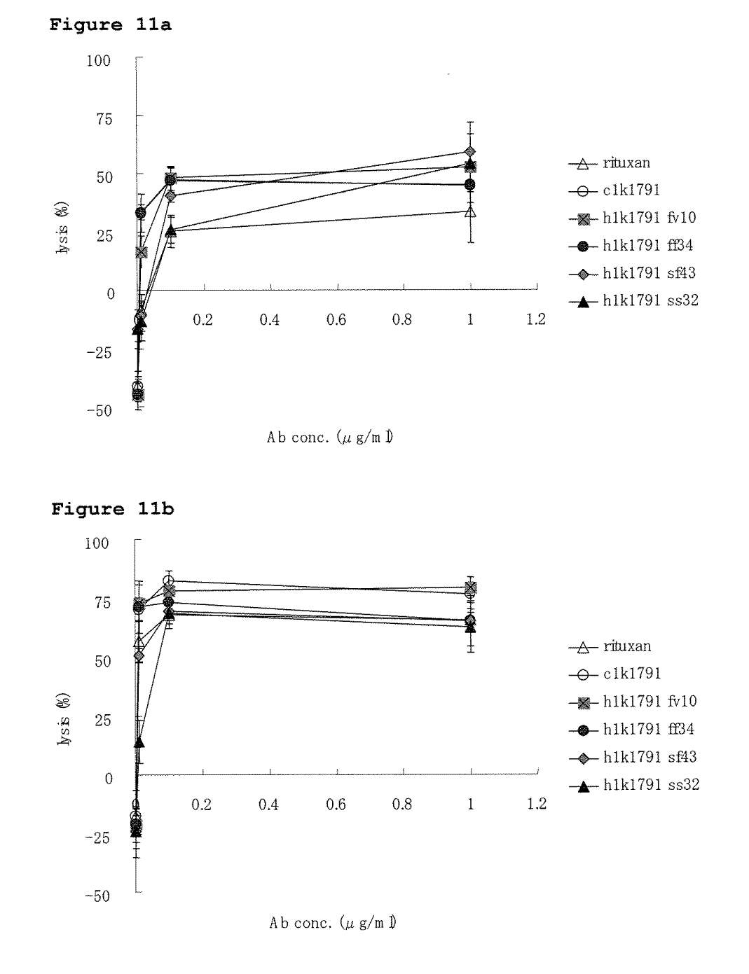

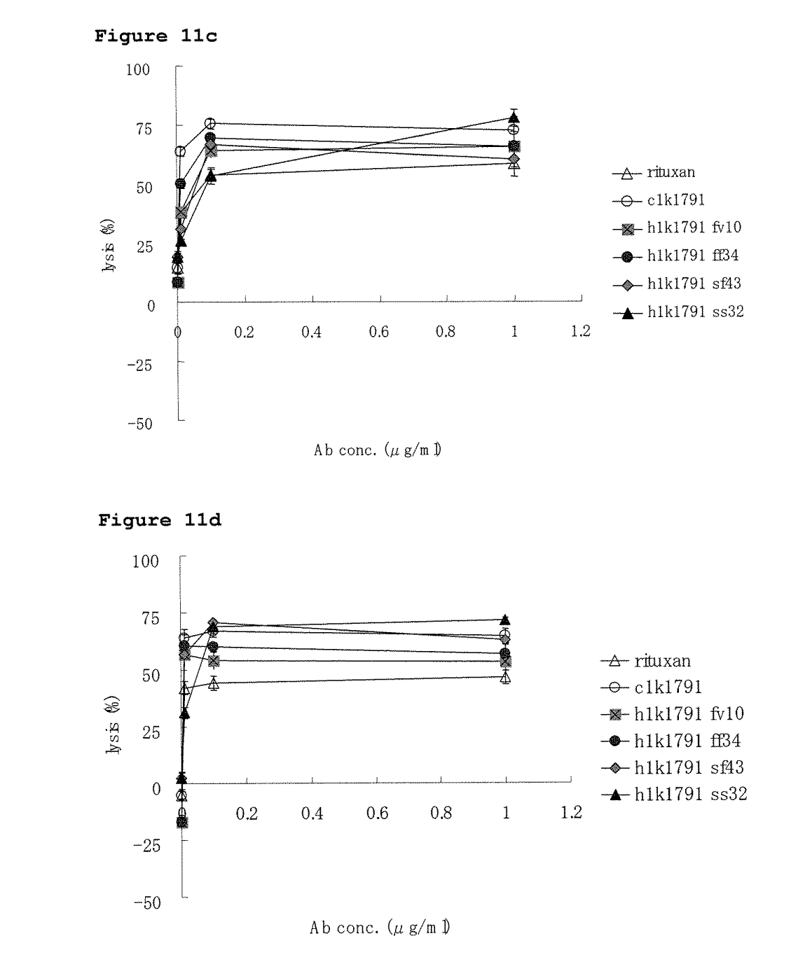

The ADCC activity of iv, ff, sf, and ss against Raji, SU-DHL4, WiL-2, and RCK8 cells was also examined. The relationship between antibody concentration and ADCC is shown in FIG. 11a to FIG. 11d (the E:T ratio was 25). ADCC activities of four clones fv, ff, sf, and ss of humanized antibody 1791 according to the present invention were equal to or greater than that of Rituxan (C2B8). The results for these ADCC activities also demonstrate the efficacy of the series 1791 of humanized antibodies according to the present invention.

### Example 4

Obtaining of Another Type of Humanized Anti-CD20 Monoclonal Antibody (Humanized Antibody 1782)

The series 1782 was selected as antibodies having highest affinities from the group of antibodies which do not induce apoptosis by themselves and have high affinity, and subjected to humanization. Clones of humanized antibody 1782 were obtained and their properties were examined in accordance with the procedures described in the previous Examples. Properties of the resulting humanized antibodies were also were examined in accordance with the procedures described in the previous Examples.

DNAs with codons optimized for CHO cells, based on amino acid sequences in SEQ ID Nos: 27 to 34, were designed and synthesized in routine procedures. Sixteen types of expression constructs for humanized 1K1782 were constructed using pNOW as an expression vector. These constructs were as follows:

pNOW-aa1782 kg1, pNOW-af1782 kg1, pNOW-as1782 kg1, pNOW-av1782 kg1, pNOW-fa1782 kg1, pNOW-ff1782 kg1, pNOW-fs1782 kg1, pNOW-fv1821 kg1, pNOW-sa1782 kg1, pNOW-sf1821 kg1, pNOW-ss1782 kg1, pNOW-sv1782 kg1, pNOW-va1782 kg1, pNOW-vf1782 kg1, pNOW-vs1782 kg1, and pNOW-vv1782 kg1.

In addition, transfection and selection using a chemical reagent were performed in accordance with the procedures described in Example 3, for selection of high expression cell strains, small scale culturing, and culturing of humanized antibody producing strains and purification of humanized antibodies.

The antibody producing strains used were CHO cells hz1782-ss21, hz1782-fv02, hz1782-ff14, and hz1782-sf23, of which the strains hz1782-ss21, hz1782-fv02, and hz1782-ff14 were deposited under Accession Nos. FERN ABP-10907, FERM ABP-10906, and FERN ABP-10905, respectively, with the International Patent Organism Depositary, National Institute of Advanced Industrial Science and Technology, Tsukuba Central 6, 1-1-1 Higashi, Tsukuba, Ibaraki, Japan, on 31 August in 19th year of Heisei (2007) under the provisions of the Budapest Treaty.

The amino acid sequences of the H-chain V-region and the L-chain V-region of humanized anti-CD20 monoclonal antibodies obtained from these cell strains (SEQ ID Nos: 17 to 24) are described hereinafter.

Sequences for Humanized 1k1782:

The sequence of the L-chain V-region (SEQ ID No: 27):

Ven 1782: DILLTQSPATLSLSPGERATLSC RASQNIGTSIH
WYQQKPGQSPRLLIK YASESFS
GIPSRFSGSGSGTDFTLTISSLEPEDFADYYC QQSNSWPFT
FGSGTKLEIK

The sequence of the H-chain V-region (SEQ ID No: 31):

Ven 1782: QVQLVQSGAELKKPGASVKVSCKASGYTFT SYWMH
WVKQAPGQGLEWIG YITPSTGYTDYNKKFKD
KATLTADRSSSTAYMEISSLRSEDTAVYYCAR SGPYFDV
WGAGTTVTVSS

The sequence of the L-chain V-region (SEQ ID No: 28):

abb 1782: DIVLTQSPATLSLSPGERATLSC RASQNIGTSIH
WYQQKPGQSPRLLIK YASESFT
GIPSRFSGSGSGTDFTLTISSLEPEDFADYYC QQSNSWPFT
FGSGTKLEIK

The sequence of the H-chain V-region (SEQ ID No: 32):

Abb 1782: QVQLVQSGAEVKKPGASVKVSCKASGYTFT SYWMH
WVRQAPGQGLEWMG YITPSTGYTDYNQKFQG
RVTLTADRSSSTAYMELSSLRSEDTAVYYCAR SGPYFDV
WGAGTTVTVSS

The sequence of the L-chain V-region (SEQ ID No: 29):

sdr 1782: DILLTQSPATLSLSPGERATLSC RASQNVGTSLH
WYQQKPGQSPRLLIK YASERFT
GIPSRFSGSGSGTDFTLTISSLEPEDFADYYC QQSNSWPFT
FGSGTKLEIK

The sequence of the H-chain V-region (SEQ ID No: 33):

sdr 1782: QVQLVQSGAEVKKPGASVKVSCKASGYTFT SYWMH
WVKQAPGQGLEWIG YITPSTGYTDYNQKFQG
KATLTADRSSSTAYMELSSLRSEDTAVYYCAR SGPYFDV
WGAGTTVTVSS

The sequence of the L-chain V-region (SEQ ID No: 30):

fra 1782: DILLTQSPATLSVSPGERATLSC RASQNIGTSIH
WYQQRPGQSPRLLIK YASESFS
GIPSRFSGSGSGTDFTLTINSLQPEDIADYYC QQSNSWPFT
FGSGTKLEIK

The sequence of the H-chain V-region (SEQ ID No: 34):

fra 1782: QVQLVQSGAEVKKPGASVKVSCKASGYTFT SYWMH
WVKQAPGQGLEWIG YITPSTGYTDYNKKFKD
KATLTADRSSSTAYMELSSLRSEDTAVYYCAR SGPYFDV
WGAGTTVTVSS

In the specification, when clone fv of humanized antibody 1782 is referred to, it means an antibody clone in which the L-chain of fra1782 (the L-chain set forth in SEQ ID No: 30) and the H-chain of Ven1782 (the H-chain set forth in SEQ ID No: 31) are combined. This similarly applies to other clones.

### (a) Measurement of Affinity of Humanized Antibody 1782

With reference to the results in Example 3, the inventors made the assumption that clones of humanized antibody 1782 are also classified into four groups, and selected ss, sf, ff, and fv belonging to the respective groups. Kd values against CD20 were measured for these antibodies, Rituxan (c2B8), and humanized antibody 2f2. The results are shown in Table 8.

In the specification, for example, when clone sf of humanized antibody 1782 is referred to, it means an antibody clone in which the L-chain of sdr1782 (the L-chain set forth in SEQ ID No: 29) and the H-chain of fra1782 (the H-chain set forth in SEQ ID No: 34) are combined (underlining is done for explanation). This similarly applies to other clones.

### Binding Dissociation Constants (Kd) of Humanized Antibody-1782 Clones

TABLE 8

| | Clone | | | | | |
|---|---|---|---|---|---|---|
| | c2B8 | 2f2 | h1782ff | h1782fv | h1782sf | h1782ss |
| Kd (nM) | 5.43 ± 0.93 | 5.26 | 4.38 | 6.76 | 7.43 | 8.64 |

The Kd values of these humanized antibodies were examined in accordance with the previous Examples. The cell used was Raji cell. Detection of the primary antibody used FITC-labeled Protein A, and analysis was made on the assumption that the binding ratio of primary antibody and Protein A is 1:3.

The clone ff of humanized antibody 1782 displayed the lowest Kd value, which was lower than those of Rituxan and humanized antibody 2f2. That is, it was found that the clone ff of humanized antibody 1782 has the highest affinity to CD20.

### (b) Cell Growth Inhibiting Activity of Humanized Antibody 1782

Four clones fv, ff, sf, and ss of humanized antibody 1782 were measured for apoptosis inducing activity, CDC activity (%), and ADCC activity. These activities were measured in accordance with the procedures described in the previous Examples.

(b1) Apoptosis Inducing Activity of Humanized Antibody

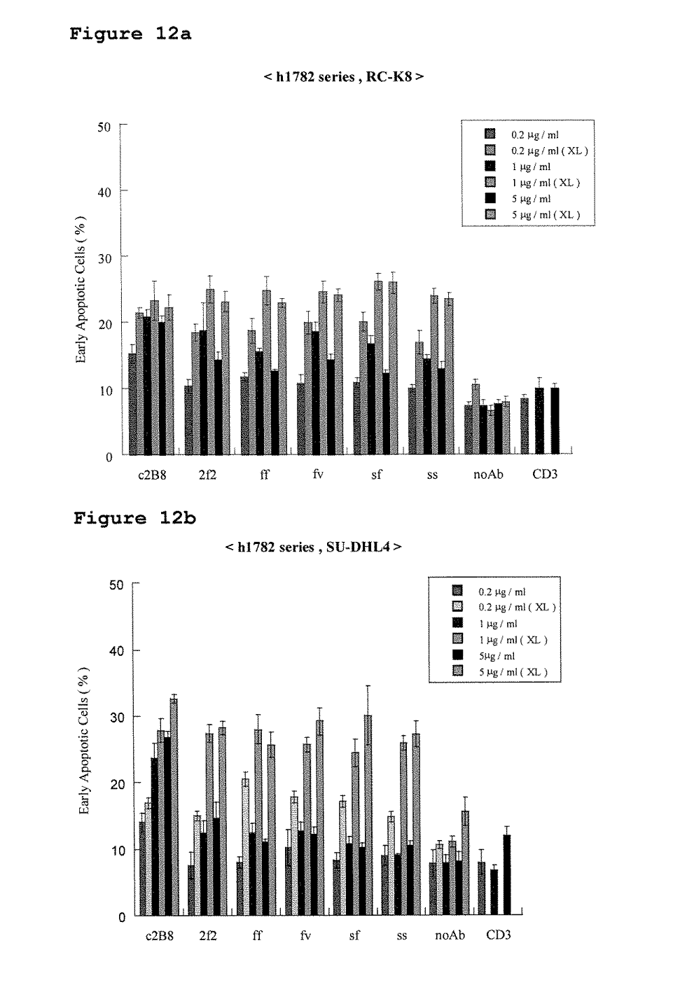

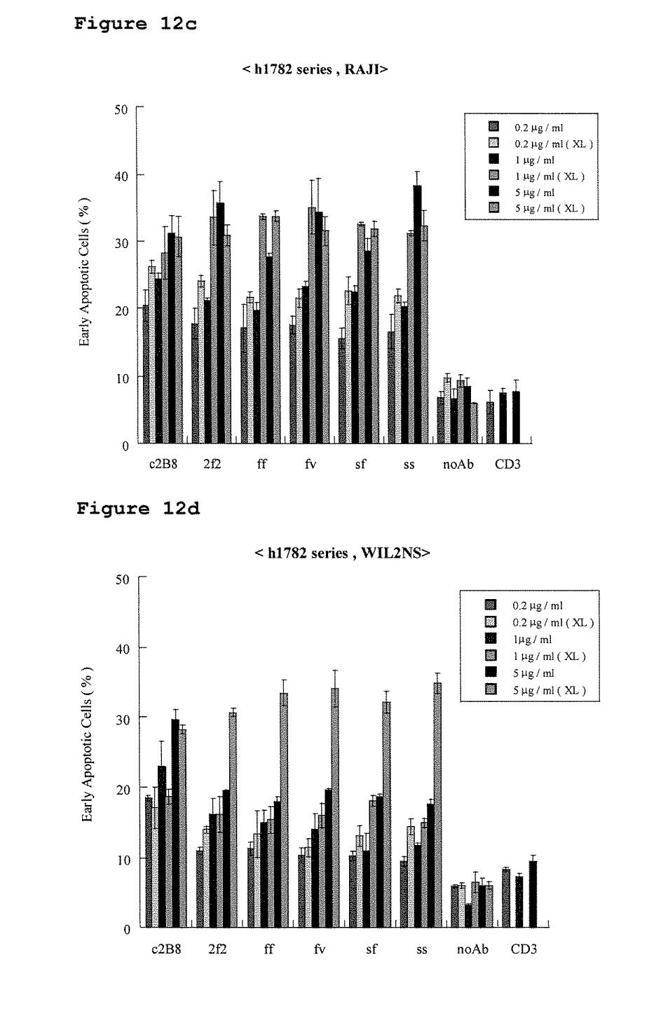

The apoptosis inducing activity of humanized antibody 1782 was measured using RC-K8, SU-DHL4, RAJI, and WIL2NS cells. The results are shown in FIG. 12a to FIG. 12d (in the figures), the numbers on the right side represent the antibody concentration, and XL represents the presence of crosslinking with a secondary antibody (goat anti-human antibody).

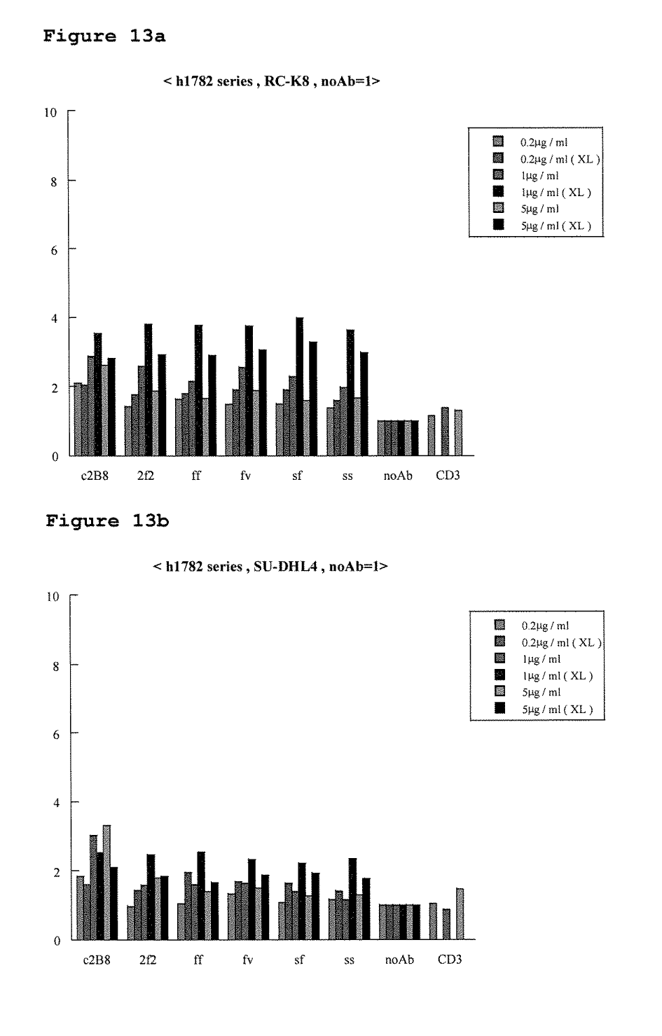

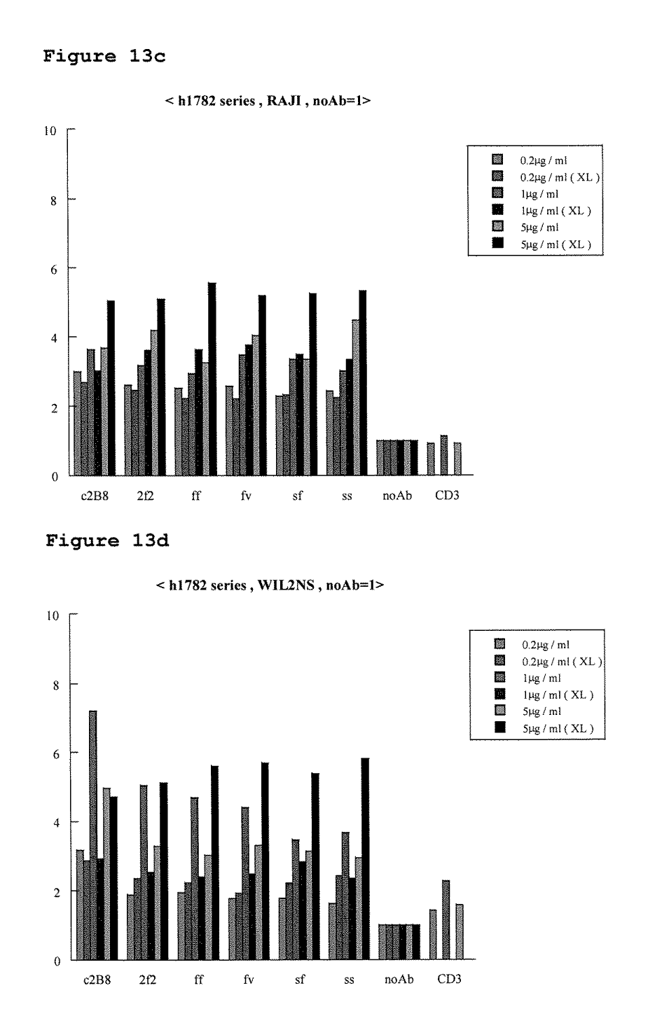

FIG. 13a to FIG. 13d show apoptosis inducing activities of humanized antibody-1782 clones, when the capability of inducing apoptosis under conditions with no added antibody is set to 1.

The series 1782 of humanized antibodies displayed weak activities of inducing apoptosis by themselves and apoptosis inducing activities comparable to or greater than that of Rituxan (c2B8) under the crosslinking condition. The results are summarized in Table 9.

Apoptosis Inducing Activities of Humanized Antibody-1782 clones (in the table, XL represents the presence of crosslinking with a secondary antibody (goat anti-human antibody).

TABLE 9

| | Affinity | Apoptosis (5 ug/ml) | | | | | | | |
|---|---|---|---|---|---|---|---|---|---|
| | (average) | RAJI | | WIL2NS | | SUDHL4 | | RC-K8 | |
| | Raji | c2B8 = | XL | c2B8 = | XL | c2B8 = | XL | c2B8 = | XL |
| Clone | Kd (nM) | 100 | w/wo | 100 | w/wo | 100 | w/wo | 100 | w/wo |
| c2B8 | 5.43 ± 0.93 | 100 | 1.0 | 100 | 1.0 | 100 | 1.2 | 100 | 1.1 |
| 2f2 | 5.26 | 115 | 0.9 | 66 | 1.6 | 54 | 1.9 | 72 | 1.6 |
| ff | 4.38 | 89 | 1.2 | 63 | 1.9 | 41 | 2.3 | 63 | 1.8 |
| fv | 6.76 | 110 | 0.9 | 61 | 1.7 | 46 | 2.4 | 72 | 1.7 |
| sf | 7.43 | 92 | 1.1 | 59 | 1.7 | 38 | 2.9 | 61 | 2.1 |
| ss | 8.64 | 123 | 0.9 | 66 | 2.0 | 39 | 2.6 | 64 | 1.8 |
| no Ab | | 27 | 0.7 | 20 | 1.0 | 30 | 1.9 | 38 | 1.0 |

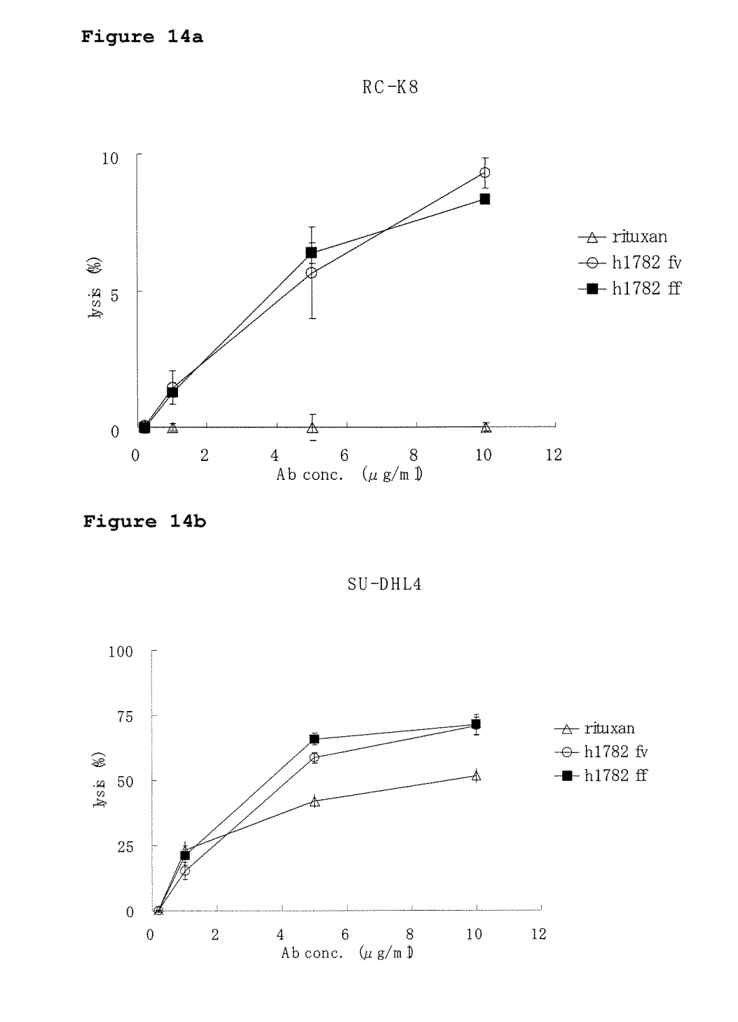

(b2) CDC Activity of Humanized Antibody 1782
CDC activities of clones fv and ff of humanized antibody 1782 were first measured using RC-K8 and SU-DHL4 cells. The results are shown in FIG. 14a and FIG. 14b. It was found that there was not much difference between activities at antibody concentrations of 0.1 and 1 μg/ml. The clones fv and ff of humanized antibody 1782 displayed high CDC activities against either of these cell types. In the case of RC-K8 cells which are resistant to Rituxan, fv had the highest CDC activity. In the case of SU-DHL4 cells, the clones fv and ff of humanized antibody 1782 displayed high CDC activities, which were greater than that of Rituxan.

CDC activities of four clones fv, ff, sf, and ss of humanized antibody 1782 were measured using RAJI, WIL2NS, SU-DHL4, and RC-K8 cells. The results are summarized in Table 10.

### CDC Activities of Humanized Antibody-1782 Clones

TABLE 10

| | CDC (10 ug/ml) | | | |
|---|---|---|---|---|
| | | | | RC-K8 |
| | RAJI | WIL2NS | SUDHL4 | h1791 |
| Clone | c2B8 = 100 | c2B8 = 100 | c2B8 = 100 | sf43 = 100 |
| c2B8 | 100 | 100 | 100 | 0 |
| ff | 94 | 68 | 138 | 19 |
| fv | 92 | 90 | 137 | 21 |
| sf | 75 | 28 | 102 | 8 |
| ss | 77 | 52 | 109 | 15 |
| h1791sf43 | | | | 100 |

These humanized antibody-1782 clones displayed CDC activities against RC-K8 cells, which are resistant to Rituxan, as well as against RAJI, WIL2NS, and SU-DHL4 cells. In particular, the clones ff and fv had high CDC activities and displayed CDC activities against SU-DHL4 cells significantly greater than that of Rituxan.

Putting together the results of Kd values and CDC activities, the clone ff is preferable in that it has the highest affinity, a relatively high CDC activity, and a CDC activity also against the strain RC-K8, a Rituxan-resistant strain, and the clone fv is preferable in that it has the highest CDC activity and in addition, is also effective against the strain RC-K8, a Rituxan-resistant strain. In particular, since the clone ff has a very high affinity, it is possible to R1 label it to employ it for missile therapy of B-cell involving diseases.

(b3) ADCC Activity of Humanized Antibody 1782

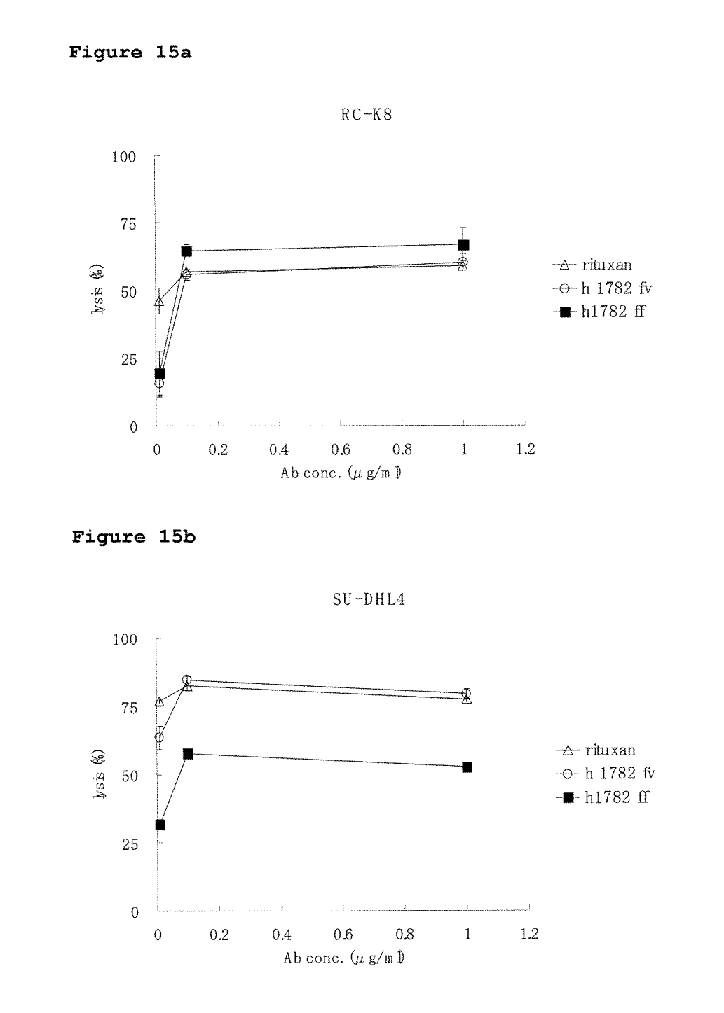

ADCC activities of the clones fv and ff of humanized antibody 1782 were first measured using RC-K8 and SU-DHL4 cells. The results are shown in FIG. 15a and FIG. 15b. It was found that there was not much difference between activities at antibody concentrations of 0.1 and 1 μg/ml. In the case of RC-K8 cells, the clone ff had a greater ADCC activity than the clone fv and Rituxan. In the case of SU-DHL4 cells, the clone fv displayed a comparable ADCC activity to that of Rituxan, while the clone ff had a relatively low ADCC activity.

ADCC activities of the four clones fv, ff, sf, and ss of humanized antibody 1782 were measured using RAJI, WIL2NS, SU-DHL4, and RC-K8 cells. The results are summarized in Table 11.

### ADCC Activities of Humanized Antibody-1782 Clones

TABLE 11

| | ADCC (25:1 1 ug/ml) | | | |
|---|---|---|---|---|
| | RAJI | WIL2NS | SUDHL4 | RC-K8 |
| Clone | c2B8 = 100 | c2B8 = 100 | c2B8 = 100 | c2B8 = 100 |
| c2B8 | 100 | 100 | 100 | 100 |
| ff | 106 | 65 | 68 | 113 |
| fv | 93 | 103 | 103 | 102 |
| sf | 109 | 99 | 70 | 144 |
| ss | 88 | 87 | 97 | 135 |

The clone fv is preferable in that it has a high ADCC activity against SU-DHL4 cell, and the clone ss is preferable in that it has a high ADCC activity against RC-K8 cells.

### INDUSTRIAL APPLICABILITY

According to the present invention, there are provided anti-CD20 monoclonal antibodies, in particular, humanized antibodies, methods of producing the same, and therapeutic agents for the treatment of B-cell involving diseases containing the same, which have high binding affinities to human CD20 molecules in their natural state and display biological activities suitable as pharmaceutical use. Therefore, the present invention is applicable in the fields of manufacturing of drugs for treating cancers, cancer research, and others.

### FREE TEXTS IN SEQUENCE LISTING

SEQ ID No: 17: L-chain V-region sequence of humanized antibody abb 1791
SEQ ID No: 18: L-chain V-region sequence of humanized antibody fra 1791
SEQ ID No: 19: L-chain V-region sequence of humanized antibody sdr 1791
SEQ ID No: 20: L-chain V-region sequence of humanized antibody Ven 1791
SEQ ID No: 21: h-chain V-region sequence of humanized antibody abb 1791
SEQ ID No: 22: H-chain V-region sequence of humanized antibody fra 1791
SEQ ID No: 23: H-chain V-region sequence of humanized antibody sdr 1791
SEQ ID No: 24: H-chain V-region sequence of humanized antibody Ven 1791
SEQ ID No: 25: primer
SEQ ID No: 26: primer
SEQ ID No: 27: L-chain V-region sequence of humanized antibody Ven 1782
SEQ ID No: 28: L-chain V-region sequence of humanized antibody abb 1782
SEQ ID No: 29: L-chain V-region sequence of humanized antibody sdr 1782
SEQ ID No: 30: L-chain V-region sequence of humanized antibody fra 1782
SEQ ID No: 31: H-chain V-region sequence of humanized antibody Ven 1791
SEQ ID No: 32: H-chain V-region sequence of humanized antibody abb 1782
SEQ ID No: 33: H-chain V-region sequence of humanized antibody sdr 1782
SEQ ID No: 34: H-chain V-region sequence of humanized antibody fra 1791
[Sequence Listing]

1. A humanized anti-CD20 monoclonal antibody, comprising a combination of the L-chain set forth in any one of SEQ ID Nos: 27 to 30 and the H-chain set forth in any one of SEQ ID Nos: 31 to 34.

2. The humanized anti-CD20 monoclonal antibody according to claim 1, comprising a combination of the L-chain set forth in SEQ ID No: 30 and the H-chain set forth in SEQ ID No: 34.

3. The humanized anti-CD20 monoclonal antibody according to claim 1, comprising a combination of the L-chain set forth in SEQ ID No: 30 and the H-chain set forth in SEQ ID No: 31.

4. The humanized anti-CD20 monoclonal antibody according to claim 1, comprising a combination of the L-chain set forth in SEQ ID No: 29 and the H-chain set forth in SEQ ID No: 33.

5. The humanized anti-CD20 monoclonal antibody according to claim 1, which is produced by a cell selected from the group consisting of cells which have been deposited under the accession numbers FERM ABP-10907, FERM ABP-10906, and FERM ABP-10905, with the International Patent Organism Depositary of the National Institute of Advanced Industrial Science and Technology.

6. A method for producing a humanized anti-CD20 monoclonal antibody, which comprises culturing a cell selected from the group consisting of cells which have been deposited under the accession numbers FERM ABP-10907, FERM ABP-10906, and FERM ABP-10905, with the International Patent Organism Depositary of the National Institute of Advanced Industrial Science and Technology.

7. A therapeutic agent for the treatment of B-cell mediated diseases, comprising as an active ingredient the humanized anti-CD20 monoclonal antibody according to claim 1.
